SEQUENCE LISTING (9 sequences)

SEQ ID NO: 1 - DNA, 2347 bp, Human
ggagttgcttgatgtgagataaatggacgtaagatttttccacctccagcccagcccgccgctgcgcccgacgctccctgtctgggaccttctccctgcctggacccctactattgcaacaagtttgacggtgagaacatgtatatgagcatgacagagccgagccaggactatgtgccagccagccagtcctaccctggtccaagcctggaaagtgaagacttcaacattccaccaattactcctccttccctccaagaccactcgctggtgcacctgaatgaagttgagtctggttaccattctctgtgtcaccccatgaaccataatggcctgctaccatttcatccacaaaacatggacctccctgaaatcacagtctccaatatgctgggccaggatggaacactgctttctaattccatttctgtgatgccagatatacgaaacccagaaggaactcagtacagttcccatcctcagatggcagccatgagaccaaggggccagcctgcacagatcaggcagcagccaggaatgatgccacatggccagctgactaccattaaccagtcacagctaagtgctcaacttggtttgaatatgggaggaagcaatgttccccacaactcaccatctccacctggaagcaagtctgcaactccttcaccatccagttcagtgcatgaagatgaaggcgatgatacctctaagatcaatggtggagagaagcggcctgcctctgatatggggaaaaaaccaaaaactcccaaaaagaagaagaagaaggatcccaatgagccccagaagcctgtgtctgcctatgcgttattctttcgtgatactcaggccgccatcaagggccaaaatccaaacgctacctttggcgaagtctctaaaattgtggcttcaatgtgggacggtttaggagaagagcaaaaacaggtctataaaaagaaaaccgaggctgcgaagaaggagtacctgaagcaactcgcagcatacagagccagccttgtatccaagagctacagtgaacctgttgacgtgaagacatctcaacctcctcagctgatcaattcgaagccgtcggtgttccatgggcccagccaggcccactcggccctgtacctaagttcccactatcaccaacaaccgggaatgaatcctcacctaactgccatgcatcctagtctccccaggaacatagcccccaagccgaataaccaaatgcaagtgactgtctctatagcaaacatggctgtgtcccctcctcctcccctccagatcagcccgcctcttcaccagcatctcaacatgcagcagcaccagccgctcaccatgcagcagccccttgggaaccagctccccatgcaggtccagtctgccttacactcacccaccatgcagcaaggatttactcttcaacccgactatcagactattatcaatcctacatctacagctgcacaagttgtcacccaggcaatggagtatgtgcgttcggggtgcagaaatcctcccccacaaccggtggactggaataacgactactgcagtagtgggggcatgcagagggacaaagcactgtaccttacttgagaatctgaacacctcttctttccactgaggaattcagggaagtgttttcaccatggattgctttgtacagtcaaggcagttctccattttattagaaaatacaagttgctaagcacttaggaccatttgagcttgtgggtcacccactctggaagaaatagtcatgcttctttattatttttttaatcctttatggacattgtttttcttcttccctgaaggaaatttggaccattcagattttatgttggttttttgctgtgaagtgctgcgctctagtaactgccttagcaactgtagatgtctcggataaaagtcctggattttccattggttttcataatgggtgtttatatgaaactactaaagactttttaaatggcttgatgtagcagtcatagcaagtttgtaaatagcatctatgttacactctcctagagtataaaatgtgaatgtttttgtagctaaattgtaattgaaactggctcattccagtttattgatttcagaataggggttaaattggcaaacattcatatttttacttcatttttaaaacaactgactgatagttctatattttcaaaatatttgaaaataaaaagtattcccaagtgattttaatttaaaaacaaattggctttgtctcattgaccaaaaaaaaaaaaagaaataaaaaaaaaaaaaggggcacctgcccgggggggcggctcgagccatagtggagcgtattaggatgggagcc

SEQ ID NO: 2 - protein, 487 aa, Human [sequence omitted]

SEQ ID NO: 3 - protein, 273 aa, Human
MYMSMTEPSQDYVPASQSYPGPSLESEDFNIPPITPPSLQDHSLVHLNEVESGYHSLCHPMNHNGLLPFHPQNMDLPEITVSNMLGQDGTLLSNSISVMPDIRNPEGTQYSSHPQMAAMRPRGQPAQIRQQPGMMPHGQLTTINQSQLSAQLGLNMGGSNVPHNSPSPPGSKSATPSPSSSVHEDEGDDTSKINGGEKRPASDMGKKPKTPKKKKKKDPNEPQKPVSAYALFFRDTQAAIKGQNPNATFGEVSKIVASMWDGLGEEQKQVYKK

SEQ ID NO: 4 - protein, 15 aa, Human
PSVFHGPSQAHSALY

SEQ ID NO: 5 - DNA, 1968 bp, Human
aggcccctgcctgccccagcatcccctgcgcgaagctgggtgccccggagagtctgaccaccatgccacctcctcgcctcctcttcttcctcctcttcctcacccccatggaagtcaggcccgaggaacctctagtggtgaaggtggaagagggagataacgctgtgctgcagtgcctcaaggggacctcagatggccccactcagcagctgacctggtctcgggagtccccgcttaaacccttcttaaaactcagcctggggctgccaggcctgggaatccacatgaggcccctggccatctggcttttcatcttcaacgtctctcaacagatggggggcttctacctgtgccagccggggcccccctctgagaaggcctggcagcctggctggacagtcaatgtggagggcagcggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagccaggacctcaccatggcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatggagacgggtctgttgttgccccgggccacagctcaagacgctggaaagtattattgtcaccgtggcaacctgaccatgtcattccacctggagatcactgctcggccagtactatggcactggctgctgaggactggtggctggaaggtctcagctgtgactttggcttatctgatcttctgcctgtgttcccttgtgggcattcttcatcttcaaagagccctggtcctgaggaggaaaagaaagcgaatgactgaccccaccaggagattcttcaaagtgacgcctcccccaggaagcgggccccagaaccagtacgggaacgtgctgtctctccccacacccacctcaggcctcggacgcgcccagcgttgggccgcaggcctggggggcactgccccgtcttatggaaacccgagcagcgacgtccaggcggatggagccttggggtcccggagcccgccgggagtgggcccagaagaagaggaaggggagggctatgaggaacctgacagtgaggaggactccgagttctatgagaacgactccaaccttgggcaggaccagctctcccaggatggcagcggctacgagaaccctgaggatgagcccctgggtcctgaggatgaagactccttctccaacgctgagtcttatgagaacgaggatgaagagctgacccagccggtcgccaggacaatggacttcctgagccctcatgggtcagcctgggaccccagccgggaagcaacctccctggcagggtcccagtcctatgaggatatgagaggaatcctgtatgcagccccccagctccgctccattcggggccagcctggacccaatcatgaggaagatgcagactcttatgagaacatggataatcccgatgggccagacccagcctggggaggagggggccgcatgggcacctggagcaccaggtgatcctcaggtggccagcctggatctcctcaagtccccaagattcacacctgactctgaaatctgaagacctcgagcagatgatgccaacctctggagcaatgttgcttaggatgtgtgcatgtgtgtaagtgtgtgtgtgtgtgtgtgtgtgtatacatgccagtgacacttccagtcccctttgtattccttaaataaactcaatgagctcttccaatcctaaaaaaaaaaa

SEQ ID NO: 6 - protein, 557 aa, Human
MPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKRKRMTDPTRRFFKVTPPPGSGPQNQYGNVLSLPTPTSGLGRAQRWAAGLGGTAPSYGNPSSDVQADGALGSRSPPGVGPEEEEGEGYEEPDSEEDSEFYENDSNLGQDQLSQDGSGYENPEDEPLGPEDEDSFSNAESYENEDEELTQPVARTMDFLSPHGSAWDPSREATSLAGSQSYEDMRGILYAAPQLRSIRGQPGPNHEEDADSYENMDNPDGPDPAWGGGGRMGTWSTR

SEQ ID NO: 7 - protein, 273 aa, Human
MPPPRLLFFLLFLTPMEVRPEEPLVVKVEEGDNAVLQCLKGTSDGPTQQLTWSRESPLKPFLKLSLGLPGLGIHMRPLASWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCVPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLE

SEQ ID NO: 8 - DNA, 18 bp, Artificial Sequence
  sTRAIL Forward cloning primer
agagaaagaggtcctcag

SEQ ID NO: 9 - DNA, 23 bp, Artificial Sequence
  sTRAIL Reverse cloning primer
ttggggcctttttagttggctaa

(19) United States
(12) Patent Application Publication
(10) Pub. No.: US 2012/0141505 A1
(43) Pub. Date: Jun. 7, 2012

(54) CD19-LIGAND AND USE

(75) Inventor: Fatih M. Uckun, White Bear Lake, MN (US)

(73) Assignee: Fatih M. Uckun, White Bear Lake, MN (US)

(21) Appl. No.: 13/286,085

(22) Filed: Oct. 31, 2011

Related U.S. Application Data
(60) Provisional application No. 61/409,084, filed on Nov. 1, 2010.

(51) Int. Cl. C07K 14/435; C07K 14/54; C07K 16/00; C12N 1/19; C12N 1/21; C12N 5/07; C12N 5/0781; A61K 38/17; A61K 31/7052; A61K 39/00; G01N 33/53; C07K 16/28; A61K 39/395; C40B 30/04; C12P 21/00; A61P 37/06; A61P 19/02; A61P 1/00; A61P 35/02; A61P 35/00; C12N 15/12

(52) U.S. Cl. 424/173.1; 530/350; 536/23.5; 530/351; 530/391.1; 435/254.2; 435/252.33; 435/358; 435/375; 514/21.2; 514/44 R; 424/184.1; 424/192.1; 435/7.24; 530/389.6; 514/16.6; 506/9; 514/19.6; 514/19.3; 435/69.1

(57) ABSTRACT

Provided herein are CD19-ligand (CD19-L) polypeptides and polynucleotides encoding such CD19-L polypeptides. Methods related to diagnosing and treating a disorder associated with CD19 positive B-cells in a patient using a CD19-L polypeptide are also provided.

### CROSS-REFERENCE TO RELATED APPLICATION

This application claims priority to U.S. Provisional Application No. 61/409,084 filed Nov. 1, 2010, which is hereby incorporated by reference for all purposes.

### BACKGROUND

CD19 is a 95-kDa orphan receptor molecule found on both B-cells and B-cell precursors. On mature B-cells, CD19 forms a multi-receptor complex consisting of CD19, CD21, CD81, and CD225 to reduce the threshold for BCR-mediated as well as CD40-mediated signaling events. CD19-deficient B cells have decreased capacity to proliferate in response to B-cell mitogens including lipopolysaccharide (LPS), anti-IgM antibodies, and CD40 ligation. CD19-deficient mice exhibit hypogammaglobulinemia and impaired humoral immune responses as well as reduced numbers of CD5+ B-1 B-cells. Forced expression of human CD19 gene in transgenic mice results in perturbation of B-cell development and humoral B-cell responses. Mutations in the human CD19 gene affecting the CD19 cytoplasmic domain have been associated with clinically significant antibody-deficiency syndromes.

The majority of cases of ALL appear to originate from putative developmental lesions in normal B-cell precursor clones during early phases of ontogeny and express CD19 co-receptor. Currently, the major challenge in the treatment of ALL is to salvage relapsed patients whose overall survival remains very poor despite intensive multi-agent chemotherapy. There is a need for new anti-leukemic agents capable of killing chemotherapy-resistant leukemic cells from patients with relapsed ALL. CD19 is abundantly expressed on leukemic cells from B-lineage ALL patients, but is absent on the parenchymal cells of life-maintaining non-hematopoietic organs, circulating blood myeloid and erythroid cells, T-cells as well as bone marrow stem cells, making it an attractive molecular target for biotherapy in relapsed ALL.

### SUMMARY

The cloning and characterization of a novel 54-kDa high-mobility group (HMG)-box protein as the ligand for the human pan-B cell co-receptor CD19 is described herein. CD19 ligand (CD19-L) is a CD19-specific recombinant human protein demonstrating potent anti-leukemic activity against B-lineage acute lymphoblastic leukemia (ALL), the most common form of childhood cancer and the second most common form of acute leukemia in adults. Soluble recombinant CD19-L protein exhibited exquisite specificity for the extracellular domain of CD19 and strong binding to the surface of B-lineage leukemia/lymphoma cells. Engagement of CD19 co-receptor on B-lineage ALL cells with CD19-L perturbed CD19-associated signaling network altering the expression levels of multiple genes directly involved in regulation of apoptosis, and triggered rapid apoptotic cell death in a CD19-specific manner.

The identified and characterized ligand, CD19-L is a useful tool for the diagnosis and treatment of disease associated with proliferation and/or increased activity of CD19-positive immune cells. CD19-L is particularly useful in disease characterized by proliferation of CD19+ B-cells, such as leukemia, graft versus host disease (GVHD), autoimmune disorders, rheumatoid arthritis, inflammatory bowel disease, organ transplant rejection, and is demonstrated herein to induce apoptosis in relapsed B-lineage ALL cells.

CD19-L is a T-cell antigen. It reacts with the extracellular domain of CD19 expressed on B-cells. This CD19-Ligand (of T-cell)/CD19 (of B-cell) interaction provides communication between cellular (T) and humoral (B) immunity.

The extracellular domain of CD19 is demonstrated to react with T-lineage leukemia/lymphoma cells because it binds to the CD19-L protein on the surface of T-lineage leukemia cells. The CD19 extracellular domain can therefore be used as a target for treatment of T-lineage leukemia/lymphoma. Accordingly, the invention provides therapeutic treatments based on the specific binding of CD19 antigen with CD19-Ligand. To target therapeutic agents to B-cells expressing CD19 antigen, CD19-Ligand can be used as a targeting moiety. To target therapeutic agents to T-cells expressing CD19-Ligand, the CD19 antigen can be used as a targeting moiety. The specific targeting moiety (CD19 antigen or CD19-Ligand) can be fused to a therapeutic agent as discussed herein and delivered to the appropriate B or T cell for therapeutic effect.

The amino acid sequence of CD19-L (SEQ ID NO: 2) is shown in FIG. 1, as well as a nucleic acid sequence encoding CD19-L as well as 5′ and 3′ untranslated regions (UTRs) (SEQ ID NO: 1). An identified extracellular domain (SEQ ID NO: 3) includes amino acids 1-273.

CD19-L contains no internal signal peptide to direct transport to the endoplasmic reticulum, but contains two nuclear export signals (NES) associated with unconventional ER and Golgi-independent transport pathways. CD19-L is selectively expressed in the nucleus, cytoplasm, and on the surface membrane of lymphocytes, and is particularly abundant in T-lineage cells, including thymocytes and leukemic T-cell precursors from T-lineage ALL patients. CD19-L is also expressed in B-lineage lymphoid cells at all stages of human B-cell ontogeny.

Expression vectors comprising a nucleic acid sequence (SEQ ID NO:1) encoding CD19-Ligand protein (SEQ ID NO: 2) can be used to express CD19-L in a transgenic cell. Transgenic cells expressing CD19-L can be used to produce CD19-L in vitro or in vivo, and as therapeutic agents providing CD19-L to subjects in need thereof. CD19-L, produced as a soluble recombinant protein, reacts with CD19 on B-cells and B-lineage leukemia/lymphoma cells, and is useful as a therapeutic agent to induce apoptosis of such cells, and/or as a targeting agent, targeting specific therapeutic molecules, to these cells.

In a similar manner, expression vectors comprising a nucleic acid sequence (SEQ ID NO: 5) encoding CD19 antigen (SEQ ID NO: 6) in a transgenic cells. Transgenic cells expressing CD19 can be used to produce CD19 in vitro or in vivo, and as therapeutic agents providing CD19 to subjects in need thereof. CD19 reacts with CD19-Ligand on T-cells and T-lineage leukemia/lymphoma cells, and is useful therapeutically, for example, as a targeting agent, targeting specific therapeutic molecules to these cells.

CD19-L can be produced under the control of specific promoters to control expression, or as part of a fusion molecule, for example with an Fc fragment of an immunoglobulin, with a label or tag molecule, with another therapeutic molecule, and the like.

Diagnostic assays for detecting the presence or progression of a disease characterized by CD19 expression can be based on the detection and/or quantification of CD19 by direct binding of CD19-L or a CD19-L fusion molecule, for example linked to a marker or tag. Such markers include, for example immunofluorescent, chemiluminescent, colorimetric markers, and the like markers. Affinity markers such as avidin and biotin can also be used.

In a similar manner, diagnostic assays for detecting the presence or progression of a T-cell disease characterized by CD19-L expression can be based on the detection and/or quantification of CD19-L by direct binding of CD19 antigen or a CD19 fusion molecule.

Anti-CD19 antibodies can be used in such diagnostic assays, including, for example, assays to detect CD19 expression in B-lineage cells. Such antibodies can bind, for example, CD19 and/or CD19 bound to CD19-L or the CD19-L extracellular domain, which may be present on a T-lymphoid cell, and may be in a complex with one or more co-receptor on the cell. Amplification and/or hybridization probes based on the CD19 gene sequence can be used to determine expression of CD19 antigen in lymphoid cells.

Anti-CD19-L antibodies can be used in such diagnostic assays, including, for example, detecting CD19-L expression in T-lineage cells. Such antibodies can bind, for example, CD19-L and/or CD19-L bound to CD19 or the CD19 the extracellular domain, which may be present on a B-lymphoid cell, and may be in a complex with one or more co-receptor on the cell. Amplification and/or hybridization probes based on the CD19-L gene sequence can be used to determine expression of CD19-L in lymphoid cells.

The identification of CD19-L as the natural ligand for CD19 provides a specific molecule for the treatment of disease characterized by expression of CD19, including B-cell leukemia and/or lymphoma, and particularly ALL, including relapsed and/or drug-resistant ALL. Treatment methods include administration of CD19-L polypeptide, for example as a soluble recombinant polypeptide, alone or as a fusion protein. Fusions proteins can include, for example fusion of CD19-L with additional therapeutic agents, markers, or other fusion agents, and can include for example, chemotherapeutic toxins, pseudomonas enteroxin, dipthereia toxin, heteroconjugates, radioimmunoconjugates, drug conjugates, antibodies such as anti-Cd3 antibody, and protein therapeutics, for example TNF-related apoptosis-inducing ligand (TRAIL)/CD253, and the like.

Molecular markers for diagnosis of leukemia/lymphoma are also provided. As disclosed herein, a panel of 13 genes demonstrating altered expression in leukemic cells induced by administration of CD19-L provide a set of biomarkers that may be used individually or in combination for the prediction of disease, including aggressive disease, and prediction of a subject's response to CD19L therapy. In one embodiment four genes were identified as associated with aggressive leukemic disease, and provide a panel of biomarkers that may be used individually or in combination for determining disease prognosis or aggressiveness.

The identification of the natural ligand for the CD19 co-receptor provides a specific and useful diagnostic tool for disease characterized by CD19 expression, a specific therapeutic molecule for the treatment of such disease, and biomarkers for the diagnosis, prognosis, and drug response prediction in such disease.

The binding relationship between CD19 antigen expressed on B-cells and CD19-Ligand expressed on T-cells also provides a useful mechanism for using the soluble forms of these molecules to interrupt interactions between T-cells and B-cells, for example in the treatment of autoimmune disease.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 shows the nucleic acid (SEQ ID NO: 1) and polypeptide (SEQ ID NO:2) sequences of human CD19-Ligand.

FIG. 2 shows sequence and structural comparison of CD19-L and 4 members of the TOX subfamily of HMG-box proteins. The HMG box regions indicated in grey [B] Alignment of HMG-box domains of CD19-L and TOX1, TOX2, TOX3, and TOX4. Positions and regions showing sequence differences are shaded in grey. [C] NES motifs in CD19-L aligned with homologous sequences. [D] Secondary sequence structures of CD19-L vs. TOX1 3′-UTR segments and 5′ UTR segments [E].

FIG. 3 shows steps used in preparation of purified soluble recombinant human CD19-L protein. [A] Map and restriction endonuclease sites for pFastBac HTb expression vector. [B] Multiple cloning site sequence of pFastBacHTb. The sequence of the 6× Histidine affinity tag, with spacer region and rTEV protease cleavage site underlined. Cleavage with rTEV occurs between gln and gly and is marked “**”. [C] Map and restriction endonuclease sites for PFBH-CD19-L plasmid. [D] Anti-His Western blot analysis of recombinant CD19-L after nickel column purification. [E] Coomassie blue (CB) stained gels of recombinant CD19-L obtained during purification using a Superdex 200 HR 10/30 sizing column. [F] Anti-His Western blot analysis of the fractions shown in [E]. [G] Coomassie blue stained CD19-L protein after the final purification step. [H] The affinity of purified CD19-L for CD19 protein as determined by ELISA using plates coated with CD19-L. Control wells were coated with recombinant IKAROS (IK1) protein; a 1:2000 dilution of the polyclonal anti-CD19 antibody was used after incubation of these control wells with CD19 protein.

FIG. 4 shows results of studies demonstrating selective binding of CD19-L to the extracellular domain of CD19, including (A) CD19-L pull-down experiments with human lymphoid Cell Lines; (B) CD19ECD pull-down experiments; (C) FACS histograms of wild-type DT40 (DT40WT) cells stained with biotin-labeled CD19-L and Avidin-FITC; (D) Western blot analysis of CD19 receptor expression in wild-type DT40 cells (DT40WT) as compared to DT40 clone expressing hCD19 gene under a chicken actin promoter (DT40hCD19+); (E) FACS histograms of human CD19 co-receptor positive transfected DT40hCD19+ cells stained with biotin-labeled CD19-L and Avidin-FITC; and (F1-F2-F3) Deconvolution microscopy images of primary leukemic cells from 3 B-lineage ALL patients stained with biotin-labeled soluble CD19-L protein and Avidin-FITC, where nuclear DNA was visualized with TOTO-3 staining.

FIG. 5 shows expression of CD19-L in human lymphoid cells by Northern blots of mRNA from the indicated cells (A, B) and tissues (C, D) hybridized with a CD19-L probe (upper panels) or beta-actin probe (lower panels).

FIG. 6 shows expression of CD19-L protein in human lymphoid cells. Western blot analysis (A-D3); Native CD19-L immune complexes by Western blot analysis with anti-CD19-L monoclonal antibody and FACS histograms of T-lineage ALL cells corresponding to pro-thymocyte and immature cortico-thymocyte stages of human T-cell ontogeny (E and F) and mature thymocytes (G) stained for CD4, CD5, CD7, CD8, and CD19-L. A polyclonal rabbit anti-CD19-L antibody was used for detection of CD19-L by indirect immunofluorescence. Other T-lineage antigens were stained by direct immunofluorescence using PE- or FITC-labeled monoclonal antibodies.

FIG. 7 shows surface expression of CD19-L protein on normal and leukemic T-cell precursors in FACS histograms of MOLT-3 human T-lineage ALL cells stained with a mouse monoclonal antibody against CD19-L and polyclonal goat-anti-mouse (GαM)-FITC antibody (A), and in deconvolution microscopy images of MOLT-3 cells stained with anti-CD19-L antibody plus GαM-FITC (B). Nuclear DNA was visualized with TOTO-3 staining.

FIG. 8 shows detection of CD19-L in membrane/cytoplasmic and nuclear protein fractions of human lymphoid cells by cell compartment expression of CD19-L. CD19-L Western blot analysis of NALM-6 B-lineage ALL cells and MOLT-3 cytoplasmic and nuclear fractions (A) and in confocal images of CD19-L localization in permeabilized cells stained with either control mouse IgG (B,D,F) or monoclonal anti-CD19-L antibody (C,E,G).

FIG. 9 demonstrates the effect of recombinant Human CD19-L on Regulation of Gene Expression in B-lineage ALL Cells. [A] Change in NALM-6 cell line gene expression after exposure to 100 ng/mL CD19-L for 24 hours. [B] Meta-Analysis CD19-L Down-Regulated Genes Using the Oncomine Database. Results are expressed as “Fold Difference” relative to ‘normal’ expression (T-test p-values <0.05).

FIG. 10 shows induction of apoptosis by administration of CD19-L to primary leukemia cells from relapsed B-lineage ALL patients independent of cellular resistance to other drugs. MTX: methotrexate, GEM: gemcitabine, ARA-C: cytarabine, MITO: mitoxantrone, VCR: vincristine, CAMPTO: camptothecin, 2-CDA: cladribine, FLU: fludarabine, FLU/DEX: fludarabine plus dexamethasone.

FIG. 11 shows apoptosis induced by CD19-L in primary leukemia cells independent of cellular resistance to other drugs. Quantitative flow cytometric apoptosis assays in primary leukemia cells from Relapsed B-lineage ALL Patients. MTX: methotrexate, GEM: gemcitabine, ARA-C: cytarabine, MITO: mitoxantrone, VCR: vincristine, CAMPTO: camptothecin, 2-CDA: cladribine, FLU: fludarabine.

FIG. 12 shows the nucleic acid and polypeptide sequences of human CD19 antigen.

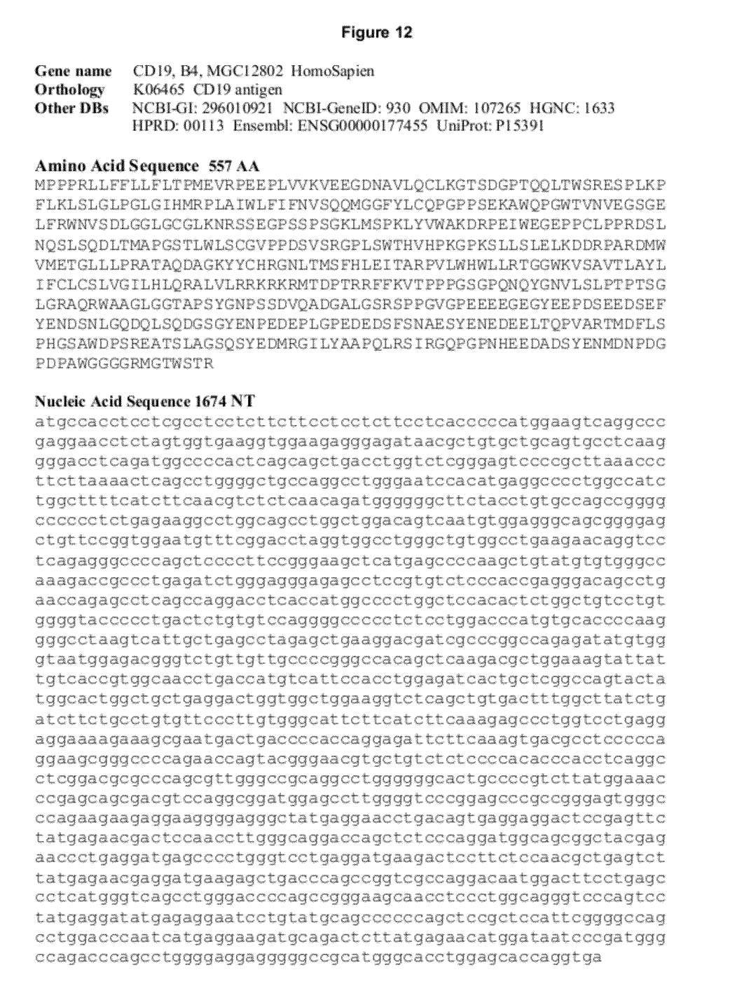

FIG. 13 shows exemplary expression constructs, including fusion constructs for expressing CD19-L fused to hIgGFc (construct 1) and CD19-L fused to sTRAIL/CD253 (construct 3).

FIG. 14 shows results of transfection of 293T cells with a fusion expression construct containing CD19-L and sTRAIL (pFUSE-CD19L:sTRAIL). One-step RT-PCR was used to demonstrate expression of a 0.5 kb PCR TRAIL product.

### DETAILED DESCRIPTION

The invention provided herein is based, in part, on the identification and characterization of a natural ligand for CD19, the human pan-B cell co-receptor, and on the use of this identified ligand in the diagnosis and treatment of disease. The disclosed CD19-Ligand is the first CD19-specific recombinant human protein identified to have potent anti-leukemic activity against B-lineage ALL.

The disclosed CD19-L polynucleotides and polypeptides are disclosed herein as useful, for example, in methods to diagnose and/or treat a subject suffering from a disorder associated with proliferation and/or increased activity of CD19 positive immune cells, including B-cells, such as acute lymphoblastic leukemia (ALL).

### CD19 and CD19-L Polypeptides

The term “CD19 polypeptide” is used herein to identify a polypeptide having the amino acid sequence of SEQ ID NO:6 and encoded by the polynucleotide sequence of SEQ ID NO:5, or variants thereof encoding the polypeptide of SEQ ID NO: 6 due to the redundancy of the genetic code. The CD19 polypeptide also includes useful fragments of CD19 antigen such as the extracellular domain (ECD) or other such fragment of SEQ ID NO:6 that retains the required ability to specifically bind the CD19-Ligand (SEQ ID NO:2) or its extracellular domain. As described herein, CD19 polypeptide is a co-receptor that binds the native receptor, CD19-Ligand (CD19-L).

The term “CD19-L polypeptide” is used herein to identify a 54 KDa high mobility group (HMG)-box polypeptide having the amino acid sequence of SEQ ID NO:2 and encoded by the coding sequence of the polynucleotide of SEQ ID NO:1, as well as variants thereof encoding the polypeptide of SEQ ID NO: 2 due to the redundancy of the genetic code. The CD19-L polypeptide also includes useful fragments of CD19-L such as the extracellular domain (ECD) (SEQ ID NO:3), a unique internal signature peptide at amino acids 317-331 (SEQ ID NO: 4) or other fragment of SEQ ID NO:2 that retains the ability to specifically bind the CD19 antigen (SEQ ID NO:6), and preferably binds the extracellular domain of CD19 when expressed on lymphoid cells.

As described herein, the CD19-L polypeptide is useful, for example, as a diagnostic molecule that selectively binds the CD19 co-receptor to diagnose the presence of disease associated with expression of CD19, such as T and B cell precursors and B-lineage lymphoid cells at all stages of human B cell ontology, including B cell lineage leukemia and lymphoma, and particularly B-cell lineage ALL.

The CD19-L polypeptide, particularly soluble CD19-L polypeptide, binds CD19 strongly binds CD19 with specificity, and to the surface of B-lineage leukemia cells expressing CD19. CD19-L is useful as a therapeutic molecule for the treatment of B-lineage leukemia/lymphomas expressing CD19. Binding of the CD19-L to the CD19 co-receptor interrupts normal CD19-mediated signaling and alters expression of genes associated with regulation of apoptosis. Treatment of CD19-expressing B-lymphoid cells with CD19-L triggers rapid apoptotic death in the treated cells, in a CD19-L specific manner.

CD19-L polypeptides, particularly ECD polypeptides, can be used to generate antibodies that specifically bind CD19-L for example, for diagnostic or purification purposes. CD19-L is also useful as a targeting moiety. When bound or fused to a second molecule, CD19-L carries the second therapeutic molecule, which may be a different therapeutic polypeptide, a radionucleotide, chemotoxic or chemostatic molecule, a biological marker or tag, or other desired molecule, to the CD19-expressing cell. Binding of CD19-L to its CD19 target permits delivery of the desired second molecule to the target cell.

### CD19 and CD19-L Polynucleotides

Polynucleotides encoding the CD19 antigen or CD19-Ligand polypeptides can be used to express the CD19 antigen or CD19-Ligand polypeptide in an expression system, for example, a cellular system. CD19-L polynucleotides can be used as probes and primers to amplify and/or hybridize CD19-L for example, for diagnosis. Useful probes and primers include those disclosed in the Examples below and similar probes and primers designed to amplify and/or detect CD19-L.

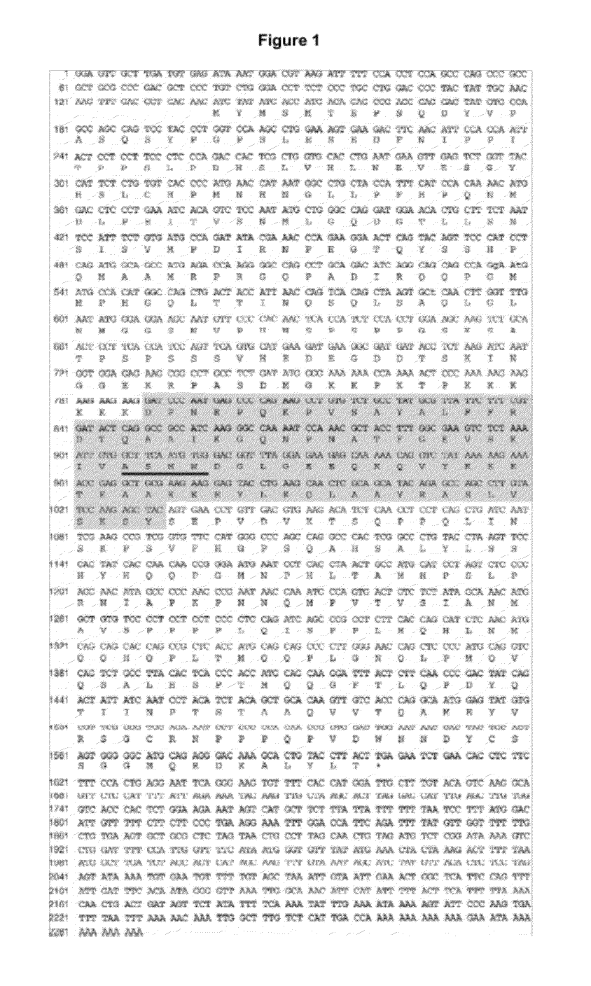

Polynucleotides encoding CD19-L can be included in an expression vector and used to express a CD19-Ligand polypeptide in cell culture, or in transformed cell provided to a patient as part of a therapeutic composition. The encoding polynucleotide can include one or both of the 5′ and 3′UTRs with the coding sequence shown in FIG. 1 (SEQ ID NO: 1), or can consist of the polynucleotide sequence of the coding region only. In one embodiment, the polynucleotide encodes the extracellular domain of CD19-L; in other embodiments, the polynucleotide encodes a fragment useful for amplification or detection of the CD19-L, or for production of specific anti-CD19L antibodies, including those disclosed in the Examples herein.

The expression vector can include additional sequences, such as promoters for controlling expression of the CD19-L polypeptide that may be heterologous promoters; signal sequences, terminator sequences, and the like. As discussed herein, CD19-L may be expressed from a fusion polynucleotide encoding other polypeptide molecules.

In one embodiment, the CD19-L polynucleotide expresses a soluble recombinant CD19-L polypeptide that can be used directly as a therapeutic or diagnostic agent.

### Fusion Polypeptide/Polynucleotide Molecules

CD19-antigen and/or CD19-Ligand polypeptide can be expressed as a fusion polypeptide from a nucleic acid encoding the polypeptide and a fusion molecule. Such fusion molecules can include, for example, additional polynucleotides or polypeptides that may be therapeutic molecules. CD19-L fusion polypeptides can be used to target the additional molecule(s) to a CD19 antigen or CD19-Ligand expressing cell.

CD19-L polypeptide can be fused with or bound by a second molecule for use in detecting CD19-L and/or CD19, directing a molecule to CD19, and or providing additional therapeutic molecules to cells expressing CD19. The second molecule can be, for example, a therapeutic molecule such as a chemotoxic or chemostatic molecule, a radionucleotide, or a therapeutic polypeptide such as tumor necrosis factor-related apoptosis-inducing ligand (TRAIL); a cytokine such as IL2, IL7, or TNF; a detection marker such as a FLAGG, poly-HIS, GST, MBP, or other tag, a chemiluminescent, immune-fluorescent, or colorimetric marker; an antibody or ligand that binds a T-cell antigen, and the like.

### Transgenic Cells

A polynucleotide encoding a CD19 antigen or CD19-Ligand polypeptide can be used to produce a transgenic cell or transgenic animal for research or for clinical purposes. In such embodiments, a polynucleotide encoding a CD19 antigen or CD19-Ligand polypeptide can be included in a vector and introduced into an animal genome.

The CD19 antigen or CD19-Ligand polynucleotide or useful fragment can be expressed, for example from know expression vectors, including bacterial, baculoviral, yeast, and the like vectors. Preferred expression is in the baculovirus expression system. Vectors can be used to transfect cells with DNA encoding CD19 antigen or CD19-Ligand, providing transgenic cells for production of CD19 antigen or CD19-Ligand in vivo or in vitro.

### Targeted Delivery

CD19 antigen or CD19-Ligand polypeptide can be linked covalently or non-covalently to a therapeutic agent to target the therapeutic agent to a cell expressing CD19 antigen or CD19-Ligand on its surface. The therapeutic agent can be a chemotherapeutic agent, for example, a chemotoxic or chemostatic compound, a radioisotope, a drug-containing nanoparticle, or a therapeutic polypeptide, for example, thymidine kinase, ricin, Pseudomonas exotoxin A, and the like.

### Methods of Diagnosis

Because of its association with immune cell disease, the presence and/or amount of CD19 antigen in a relevant sample is useful as a diagnostic marker and prognostic measurement. CD19-Ligand (CD19L) binds with specificity to CD19 antigen, and provides a useful tool for determining diagnosis or prognosis of CD19 antigen-related or CD19-Ligand-related disease. Disease characterized as expressing CD-19 includes immune B-cell disorders, for example B-lineage leukemia's and lymphomas, including B-lineage ALL.

Because of its association with immune cell disease, the presence and/or amount of CD19-Ligand in a sample is useful as a diagnostic marker and prognostic measurement. CD19-antigen (CD19) binds with specificity to CD19-Ligand, and provides a useful tool for diagnosis of CD19-Ligand related diseases. Disease characterized as expressing CD-19 includes immune T-cell disorders, for example T-lineage leukemia's and lymphomas, including T-lineage ALL.

Because the specific binding and reaction of CD19 antigen expressed on B-cells with CD19-Ligand expressed on T-cells provides a specific communication between cellular (T) and humoral (B) immunity, soluble recombinant CD19 antigen and/or soluble recombinant CD19-L polypeptide provide therapeutic molecules for disruption of T and B cell interactions. Such disruption is useful, for example, in the treatment of graft versus host disease (GVHD), autoimmune disorders, rheumatoid arthritis, inflammatory bowel disease, organ transplant rejection, including xenotransplants, and the like.

The CD19-L polypeptides described herein can be used to detect the presence of a CD19 polypeptide, for example, to detect and/or quantify expression of CD19 on B-cells and/or to detect and/or quantify CD19-expressing cells in a sample. Detection of CD19 or CD19-expressing cells in a sample obtained from a subject can be used to diagnose the presence of B-cell disease, to monitor disease progression, and/or monitor response to therapy.

In a similar manner, the CD19 antigen polypeptides described herein can be used to detect the presence of a CD19-Ligand polypeptide, for example, to detect and/or quantify expression of CD19-L on B-cells and/or to detect and/or quantify CD19-L expressing cells in a sample. Detection of CD19-L or CD19-L expressing cells in a sample obtained from a subject can be used to diagnose the presence of T-cell disease, to monitor disease progression, and/or monitor response to therapy.

For use in a diagnostic assay, CD19 antigen or CD19-Ligand polypeptide, including a fragment such as the ECD, can be linked to a detection motif such as an immunofluorescent marker, such as GFP, YFP, or RFP, linked to a marker that transforms a substrate into chemiluminescent or colorimetric signals such as luciferase, renilla-luciferase, horse radish peroxidase, or alkaline phosphatase, or linked to an affinity tag such as biotin or avidin, and the like. The CD19 antigen or CD19-Ligand polypeptide can also be linked to a fusion tag for detection. A variety of useful fusion tags are commercially available, such as a FLAG-tag (for example, DYKDDDK), poly-His tag, GST tag, MBP tag, and the like. Binding of a CD19 antigen or CD19-Ligand fusion tag to its respective binding partner can be detected and/or quantified, for example, by analysis of the linked tag. In an alternative embodiment, binding of CD19-Ligand to a CD19 target can be detected and/or quantified with anti-CD19-L antibody, for example, in an immunoassay format such as an ELISA, Western blot, and the like immunoassay. In a similar manner, binding of CD19 antigen to a CD19-Ligand target can be detected and/or quantified with anti-CD19 antibody.

In an embodiment, CD19-L can be labeled for use in diagnostic staining of CD19 expressing B-cell lineage leukemia/lymphoma cells and/or for detecting residual B-cell lineage leukemia/lymphoma cells in a subject after therapeutic treatment. In a similar manner, CD19 antigen can be labeled for use in diagnostic staining of CD19-Ligand expressing T-cell lineage leukemia/lymphoma cells and/or for detecting residual T-cell lineage leukemia/lymphoma cells in a subject after therapeutic treatment. The label may be, for example, biotin and/or an immunofluorescent, chemiluminescent, or colorimetric label, and the like, as disclosed above.

Amplification of the polynucleotide sequence encoding the CD19-Ligand, for example, by Polymerase Chain Reaction (PCR) methods provides a diagnostic method for detecting CD19-Ligand transcripts. T-cell leukemia/lymphoma can be diagnosed using such methods.

### Antibodies

CD19 antigen and CD19-Ligand polypeptides, including fragments such as the ECD, can be used as antigens to produce anti-CD19 and antiCD19-L antibodies, respectively. Such antibodies include, without limitation, monoclonal, polyclonal, antibody fragments, antibody conjugates, and humanized antibodies using known methods. In a preferred embodiment, the antibody is produced using the ECD or a portion of the ECD as an antigen.

The antibodies may detect soluble polypeptides expressed on the surface or within cells, for example, human cells. CD19-L antibodies can also be used to bind to CD19-L-bound protein complexes, for example, the multi-receptor complex present on B-cells that includes CD19. In some embodiments, the antibody can be used to separate such complexes from other proteins.

Anti-CD19-L antibodies can be used to diagnose and/or treat T-lineage lymphoid malignancies by binding to CD19-L that is bound to CD19-expressing cells. Anti-CD19 antibodies can be used to treat or and/or diagnose B-lineage lymphoid malignancies. One or both antibody may be useful to disrupt interactions between T-cells and B-cells to treat autoimmune disorders.

### Biomarkers for CD19-L Therapy

As demonstrated in the Examples below, specific biomarkers can be analyzed to determine if a subject's leukemia and/or lymphoma may be susceptible to therapeutic treatment by CD19-L. A presence or increase in anti-apoptosis related genes IFR4, MAPK11, TNFRSF7/CD27, ETS1, YY1, IRAK2, PAK5, and/or the absence or decrease in the apoptosis related genes TNFSF7, SPP1, TNFSF18, FLT3LG, HDAC5, NFKB1 is indicative of likely response to CD19-L therapy, as expression of these genes is significantly impacted by treatment with CD19-L. Methods and tools for determining the likelihood of responding to CD19-L therapy include interrogation of all or a subset of these biomarkers.

In an embodiment, a biomarker panel and methods for selecting a subject suitable for treatment of leukemia and/or lymphoma with CD19-L comprises all or a subset of the genes: TNFRSF7/CD27, IRF4, PAX5, and YY1, where the absence or decrease in expression of the genes is indicative of likely response to CD19L therapy.

### Biomarkers Indicating Aggressive Disease

As disclosed in the Examples below, results of metadata analysis provide a basis for analysis and determination of a subjects disease as a form of aggressive leukemia and/or lymphoma using tools and methods to analyze the subject's sample for increased expression of all or a subset of the following genes, CD19-L, TNFRSF7/CD27, IRF4, PAX5, and YY1, versus a normal lymphocyte control.

Methods and test kits providing amplification and/or hybridization probes for the analysis of the gene profiles discussed above are provided herein.

### Methods of Treatment

CD19-L can be administered to CD19-expressing B-lineage leukemic cells to induce apoptosis and treat CD-19 associated lymphoid disorders. Administration can be of soluble CD19-L polypeptide; of a fusion molecule comprising CD19-L, or by administration of a polynucleotide or transformed cell expressing CD19-L polypeptide or fusion molecule.

Subjects to be treated by CD19-L therapy can be identified by screening a sample obtained by the subject for the presence and/or amount of CD19-expressing B-lineage lymphoid cells. Those subjects demonstrating the presence of CD19-expressing B-lineage lymphoid cells would be selected for CD19-L therapy; those lacking such cells would not be selected. Prognosis can also be monitored during the course of treatment by analyzing the expression of CD19-expressing B-lineage lymphoid cells, with positive prognosis correlated with a decline in the presence or amount of such CD19-positive cells.

As disclosed in the Examples below, binding of CD19-L to the CD19 co-receptor on leukemic cells expressing CD19 corrupted the regulation of gene expression, altering the expression of at least 13 genes that play an active role in the regulation of apoptosis. Six of these genes upregulated (TNFSF7, SPP1, TNFSF18, FLT3LG, HDAC5, NFKB1) and seven are downregulated (IRF4, MAPK11, TNFRSF7/CD27, ETS1, YY1, IRAK2, PAX5) by treatment with CD19-L. Meta analysis showed four of the genes that are highly down-regulated by treatment with CD19-L (TNFRSF7/CD27, IRF4, PAX5, and YY1).

Selection of patients for treatment by CD19-L therapy can be determined using the identified biomarkers as discussed above and in the Examples. CD19-L and/or the additional biomarkers identified herein can also be useful in the screening of potential therapeutic drugs for efficacy in treating B-lineage leukemia/lymphoma, including therapy-resistant and aggressive forms of disease.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the above described features.

### EXAMPLES

The present invention is further exemplified by the following Examples. The examples are not intended to in any way limit the scope of the present application and its uses.

### Example 1

### Cloning and Analysis of CD19-L Sequence

A yeast two-hybrid system was used to clone the gene encoding a natural ligand, CD19-L, of human CD19 receptor. The two-hybrid “bait” construct included cDNA encoding the human CD19 extracellular domain (amino acids 1 to 273 of SEQ ID NO: 6) (CD19ECD) fused to the GAL4 DNA binding domain. CD19ECD cDNA was in frame cloned into pAS2-1 (Harper, et al., 1993, Cell. 75:805-816) at the EcoRI and Sal I sites to express CD19ECD as a fusion protein of the Gal4 DNA binding domain in yeast. A cDNA library was generated by RT-PCR using human thymus mRNA (CLONTECH) as template. A MATCHMAKER cDNA library was constructed on pGAD10 vector (CLONTECH) containing the GAL4 activation domain by ligation of cDNA library into pGAD10. Yeast strain CG1945 (Feilotter, et al., 1994, Nucleic Acids Res. 22:1502-1503) was transformed with the CD19ECD expression vector and used to screen this library.

Primary screening was conducted on Ura/Trp/Leu/His selective plates and positive clones were collected for further characterization. To eliminate false positives, second round growth assays on Ura/Trp/Leu/His selective plates and PCR analyses to confirm the presence of inserts were carried out. Library plasmids harboring the candidate CD19-L cDNA were rescued by transformation of KC8 electro-competent E-coli cells (CLONTECH) by electroporation followed by selections on M9 medium plates lack of leucine (Kaiser, et al., 1993, Bio Techniques 14:552).

The rescued plasmids containing cDNA inserts were then further characterized by sequencing. 5′ RACE and 3′ RACE experiments using a human thymus cDNA library as template were carried out to clone the missing 5′ and 3′ cDNA fragments. Full-length CD19-L cDNA was obtained by PCR reaction using KlenTaq DNA polymerase (CLONTECH) and the primers derived from the sequences of RACE clones.

One of the clones collected for further plasmid rescue based on the secondary screening on Ura/Trp/Leu/His selective plates was characterized by sequencing. The full length cDNA of this CD19-L clone was obtained by 5′/3′ RACE experiments and screening of the human thymus cDNA library. The nucleic acid sequence encoding the natural ligand of human CD19 receptor (CD19-L) cloned from a human thymus cDNA library was then determined. The cDNA for the surface membrane-associated CD19-L protein is 2290-base pairs in length terminating in a poly(A) tail (SEQ ID NO:1), and encoding a 487-amino acid protein (SEQ ID NO:2) with a predicted molecular mass of 54-kDa, as shown in FIG. 1.

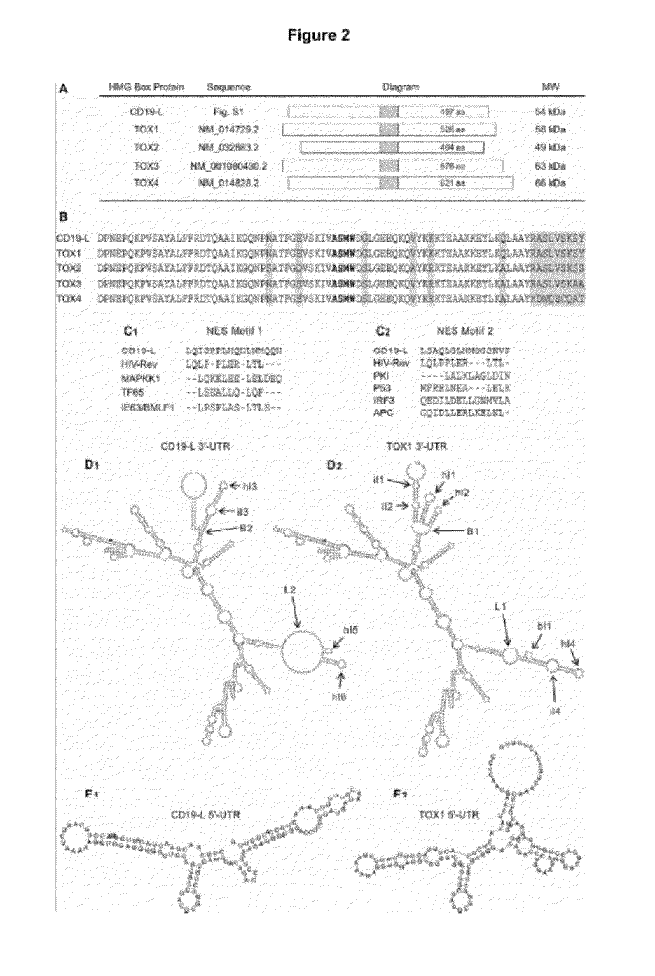

The sequence of the protein encoded by CD19-L cDNA was compared with reported sequences in Genebank Database by advanced BLAST search. The comparison revealed that CD19-L is a high mobility group boxy (HMG-box) protein with homology to portions of TOX1 (NM_{—}014729.2)/KIAA0808, TOX2 (NM_{—}032883.2), TOX3 (NM_{—}001080430.2), and TOX4 (NM_{—}014828.2). CD19-L showed the highest level of sequence similarity to the 33-amino acid larger TOX1 protein, a 58-kDa T-cell nuclear transcription factor that plays a role in the regulation of thymocyte differentiation, CD8 lineage commitment and completion of positive selection of CD4 lineage (see FIG. 2 (A,B)).

CD19-L includes a HMG-box sequence detected at nucleotides 790-1032 and encoding a single centrally located 81-amino acid segment (D217-Y298) containing the shared ASMW motif (AA 257-260) characteristic of HMG boxes.

TOX1 differs in structure and in function from CD19-L. The first 199 nucleotides of TOX1 cDNA are not found in the CD19-L cDNA. Additional differences between the 5′ CD19-L sequence and the 5′ TOX1 sequence include a 2-base pair deletion at residues 240-241 (delAT) and a 2-base pair variation at position 219-220 of the TOX1 sequence (TG>AT). Notably, the protein-coding segment of the CD19-L cDNA from residue 121 to 2257 showed 100% homology to a portion of the TOX1 protein-coding sequence. Nucleotides 790-1032 encode a 81-amino acid segment (D217 to Y298) containing an ASMW motif (amino acids 257-260) common to HMG boxes as shown in FIG. 2(B).

For several proteins, clusters of leucine and other hydrophobic residues have been shown to function as nuclear export signals (NES) that facilitate the shuttling of proteins between nucleus and cytoplasm. For export from the nucleus, nuclear/cytoplasmic secretory proteins that utilize an unconventional transport pathway can associate with other proteins containing NES or nuclear-cytoplasmic shuttling sequences. A database of experimentally validated leucine-rich NES (la Cour, et al., 2003, Nucleic Acids Res. 31:393-396) was surveyed for motifs that aligned with a putative NES in the CD19-L protein sequence. Multiple alignments were performed using the Clustal W Algorithms™ (BioEdit Sequence Alignment editor) that distinguished 2 leucine-rich motifs using sequences known to abrogate nuclear export in 9 proteins examined.

NES motifs are associated with unconventional ER- and Golgi-independent transport of nuclear/cytoplasmic secretory proteins to the surface membrane. Two NES motifs were identified in CD19-L. One motif (NES Motif 1) contains residues L382, 1384, L388 and L392 and aligns with corresponding NES sequences for HIV-Rev, MAPKK1, TF65, and IE63/BMLF1, shown in FIG. 2 (C1). The second motif (NES Motif 2) contains residues L148, L152 and L154 and aligns with NES sequences in HIV-Rev, p53, cAMP-dependent protein kinase inhibitor (PKI), interferon regulatory factor 3 (IRF3) and adenomatous polyposis coli (APC) protein shown in FIG. 2(C2).

CD19-L is expressed from a gene that differs from the gene encoding TOX1, not only in the missing coding sequences, but also in sequence and structure of the 5′ and 3′ UTRs. The untranslated regions (UTRs) of mRNAs contain regulatory elements that affect mRNA translation, stability, and transport. The secondary structures of the CD19-L 3′ and 5′ UTRs were predicted using a computational secondary structure prediction algorithm as described above and compared to the 3′ and 5′ UTR secondary structures for TOX1/KIAA0808. The sequences used to predict secondary structure for the 3′ UTRs included nucleotides T1600 to A2289 for CD19-L and nucleotides T1802 to A2493 for the TOX1 gene. The sequences used to predict secondary structure for the 5′ UTRs included nucleotides G1 to C138 for CD19-L and nucleotides G200 to C340 for the TOX1 gene.

As shown in FIGS. 2 (D and E), the results predicted strikingly different mRNA secondary structures in the UTRs of CD19-L and TOX1 mRNA. Ten hairpin loops, 3 bulges, 9 multi-branch loops and 13 internal loops were predicted for the CD19-L 3′ UTR sequence, and 10 hairpin loops, 4 bulges, 8 multi-branch loops and 16 internal loops were predicted for the TOX1 3′ UTR sequence. There were 3 distinct branches in the predicted TOX1 3′ UTR folded structure as compared to 2 distinct branches in the CD19-L 3′ UTR structure, arising from a multi-branch loop (B1 and B2) with each of these branches giving rise to different loop structures. In the TOX1 3′ UTR structure, one branch included 2 internal loops (il1, il2) and two of the branches ended in hairpin loops (hl1, hl2), while in the CD19-L 3′ UTR structure, one of the branches included an internal loop (il3) ending in a hairpin loop (hl3). In addition, internal loop L1 of the TOX1 3′ UTR was connected to a bulge (bl1) followed by an internal loop (il4) ending in a hairpin loop (hl4) in the TOX1 structure, whereas in the CD19-L structure the corresponding internal loop formed a large multi-branch loop (L2) that ended with two hairpin loops (hl5, hl6). Comparisons of the predicted secondary structures for the 3′ UTR and 5′ UTR segments are shown in FIG. 2(D) and FIG. 2(E), respectively.

The cloned CD19-L cDNA sequence and the genomic DNA sequence for Homo sapiens mRNA for KIAA0808 protein representing a partial coding sequence for the TOX1 gene (chromosome 8: 59,717,977-60,031,767 reverse strand) were converted to RNA complement sequence for alignment using the ClustalW program (BioEdit Sequence Alignment editor). Multiple alignment was constructed using gap penalties in a position- and residue-specific manner such that all pairs of sequences were aligned separately in order to calculate a distance matrix for each pair of sequences (Fast Approximate Method), then a guide tree was calculated from the distance matrix and the sequences were progressively aligned according to the branching order in the guide tree (Neighbour-Joining method).

This alignment was used to derive intronic sequences flanking the coding regions for secondary structure prediction. RNAfold, provided by the Vienna RNA package (http://rna.tbi.univie.ac.at) was used to calculate minimum free energy folding and equilibrium base-pairing probabilities for the pre-mRNA segment corresponding to the intronic RNA complement to explore how the target sequences for RNA binding proteins residing in loop structures, bulges or binding pair probabilities with low values varied between the CD19-L and TOX1 secondary structures.

### Example 2

### CD19-L Binding Analysis, Generation of Recombinant CD19-L Protein, and Production of Anti-CD19-L Antibodies

### Production of Recombinant CD19L

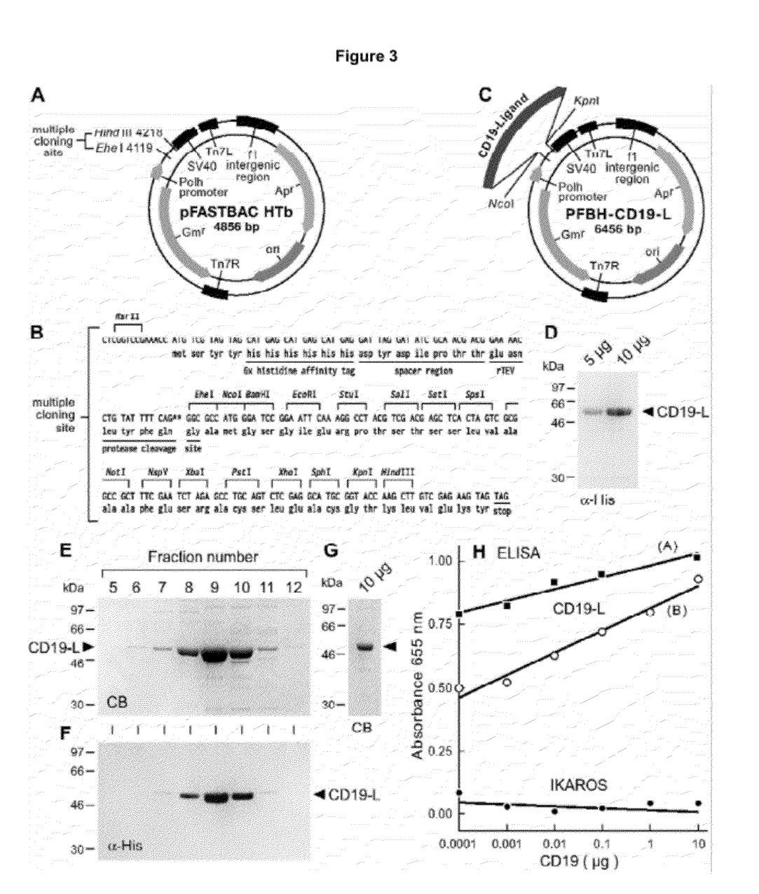

A 1.6-kb CD19-L cDNA fragment representing the protein coding segment of SEQ ID NO:1 was cloned into the NcoI/KpnI site of the 4.9-kb pFastBacHT (PFBH) donor vector (Life Technologies) containing a 6×-histidine (6×His) tag to construct a 6.5-kb recombinant PFBH-CD19-L plasmid (FIG. 3). PFBH-CD19-L was used to generate a recombinant baculovirus by site-specific transposition in Escherichia (E.) coli DH10Bac competent cells (Life Technologies) that harbor a baculovirus shuttle vector (bacmid), bMON14272 with a mini-attTn7 target site for site-specific transposition using previously reported procedures (Mahajan, et al. 2001, J. Biol Chem. 276:31216-31228; Uckun, et al. 2010, Proc. Natl. Acad. Sci. USA. 107:2902-2907).

Bacterial colonies containing recombinant bacmids were identified by disruption of the lacZa gene. High molecular weight miniprep DNA was prepared from selected E. coli clones containing recombinant bacmid and transfected into Sf21 cells using the Cellfectin reagent (Life Technologies) as previously described (Mahajan, et al. 2001, J. Biol Chem. 276:31216-31228; Uckun, et al. 2010, Proc. Natl. Acad. Sci. USA. 107:2902-2907). For large-scale production of recombinant CD19-L, 1-liter cultures of Sf21 cells in 3-liter Bellco spinner flasks (1.0-1.2×106 cells/ml) were infected with the recombinant plasmid PFBH-CD19-L (approximately 2×10e8 plaque-forming units/ml) during logarithmic growth (MOI: approximately 6).

Cultures were incubated at 28° C. and stirred at 80-100 rpm using a magnetic stirrer (Bellco Glass, Inc., Vineland, N.J.) for 48 hours Infected cells were harvested by gentle centrifugation in a Beckman GS-6 centrifuge at 500×g for 7 minutes at room temperature. Cells from 1-liter cultures were flash-frozen at −80° C. and stored at −80° C. until purification of recombinant CD19-L protein.

To extract recombinant CD19-L, frozen Sf21 insect cells previously infected with PFBH-CD19-L were lysed in 1× Triton X-100 extraction buffer (1% Triton X-100, 10 mM Tris, 130 mM NaCl, 10 mM NaF, 10 mM sodium phosphate, pH 7.5) (1 mL lysis buffer per 20×106 cells). One pellet of Complete™ protease inhibitors (Roche Molecular Biochemicals) was added for each 25 mL of the lysate and the mixture was rotated for 2 hours at 4° C. The cell pellets were centrifuged at 45,000 rpm×1 hour in a Beckman Optima LE-80K ultracentrifuge using a 45 Ti rotor. Following centrifugation, the clarified supernatant was filtered through a 0.22 μm membrane filter (S100) and dialyzed for 4 hours into buffer A (20 mM sodium phosphate, 10% glycerol, pH 7.2).

For purification of CD19-L protein, dialyzed supernatant was applied to a Nickel-chelation column (Pharmacia) that was equilibrated with buffer B (20 mM sodium phosphate, 500 mM NaCl, 0.5 M imidazole, 10% glycerol, pH 7.2). CD19-L enriched fractions were dialyzed against buffer C (20 mM Tris, pH 8.0 with 1 M DTT, 10% glycerol) overnight to remove imidazole and then applied to a Sepharose Q HP26/10 ion exchange column (column volume 50 ml; Amersham Pharmacia Biotech) followed by size exclusion chromatography on a Superdex 200 HR 10/30 column (Pharmacia) for further purification.

Excised CD19-L protein band was subjected to in situ enzymatic digestion with Lys C protease. The resulting peptide fragments were separated by reverse-phase HPLC, collected directly onto a polyvinylidine difluoride strip, treated with biobrene in methanol. The identity of a 15-amino acid internal signature peptide (PSVFHGPSQAHSALY=CD19-L AA 317-331 SEQ ID NO:4) was confirmed by microsequencing (Applied Biosystems Procise 492 HT, Applied Biosystems, Foster City, Calif.) at the Mayo Protein Core Facility (Mayo Clinic, MN).

Binding of rCD19-L to CD19

The affinity of purified CD19-L with CD19 protein was confirmed by ELISA using Nunc Immunomodule Maxisorb plates (Fischer Scientific) coated with 150 ng CD19-L in 50 μA PBS/well by overnight incubation at room temperature followed by three washes with TTBS (20 mM Tris-HCl (pH 7.5) containing 0.5 M NaCl and 0.05% Tween 20, blocking with 5% BSA in TTBS for 1 hour and three additional washes in TTBS. Plates were incubated with increasing concentrations of purified recombinant CD19 protein (0.1 ng-10 μg in 50 μl) for 2 hours at room temperature.

Following three washes with TTBS to remove unbound CD19, a rabbit polyclonal anti-CD19 antibody (1:2000 or 1:5000 dilution in 50 μl 1% BSA/TTBS) was added to each well for 60 minutes incubation at room temperature. Following three washes with TTBS, 10 ng goat anti-rabbit horseradish peroxidase-conjugated IgG in 50 μl 1% BSA/TTBS was added to each well for an additional 30 minutes incubation. After three washes with TTBS, 75 μl TMB peroxidase substrate (Biorad) was added to each well and absorbance at 655 nm was read on a Biotek EL312e plate reader after 20 minutes. Control wells were coated with recombinant IKAROS (IK1) protein (150 ng IK1 in 50 μl PBS/well) instead of CD19-L. A 1:2000 dilution of the polyclonal anti-CD19 antibody was used after treatment of these control wells with CD19 protein.

The polyclonal anti-CD19 antibody was developed in rabbits against GST-CD19 (AA 410-540) fusion protein. CD19-L protein was used to develop rabbit polyclonal and mouse monoclonal antibodies using standard immunization methods and hybridoma technology, respectively.

The cDNA encoding the extracellular domain (AA 1-273) of human CD19 (CD19ECD) (SEQ ID NO:7) was cloned into pFastBacl (PFB) donor vector (Life Technologies). The resulting pFast-bac-CD19ECD recombinant plasmid (PFB-CD19ECD) was then used to generate the recombinant baculovirus by site-specific transposition in Escherichia coli DH10Bac competent cells (Life Technologies), that harbor a baculovirus shuttle vector (bacmid), bMON14272 with a mini-attTn7 target site for site-specific transposition using known procedures (Mahajan, et al. 2001, J. Biol Chem. 276:31216-31228; Uckun, et al. 2010, Proc. Natl. Acad. Sci. USA. 107:2902-2907).

PFB-CD19ECD was inoculated into SF21 cells, and the cells were used 48 hours later for pull-down and immunoblotting experiments. Likewise, the cDNA encoding full-length CD19 as well as cDNA encoding the intracellular domain (ICD) (AA 300-540) of human CD19 were cloned into pFastBacl vector and recombinant full-length CD19 and CD191CD were produced in the baculovirus expression system. The recombinant CD19 proteins were extracted and purified using methods similar to those discussed above.

### Pull-Down Experiments Using CD19-L Protein

50 μg CD19-L protein was incubated with 50μL nickel beads (Gong, et al. 2006, Nature Structural & Molecular Biology 13:902-907) for 2 hours at 4° C. The beads were washed 3× with 1% Nonidet P-40 lysis buffer. Human T-lineage ALL cell line MOLT-3, pre-B ALL cell line NALM-6, Burkitt's leukemia/lymphoma cell lines DAUDI and RAMOS, EBV-transformed lymphoblastoid cell line BCL-1 (4×106 cells/sample) were lysed with 1% Nonidet P-40 lysis buffer and lysates were incubated with 50 μg CD19-L protein in 50 μL volume coupled with nickel beads for 2 hours on ice. Control lysates were incubated with nickel beads without coupled CD19-L protein. The protein complexes were washed with cold 1% Nonidet P-40 buffer and resuspended in reducing SDS sample buffer.

Samples were boiled for 10 minutes and fractionated on 12.5% SDS-PAGE. Proteins were transferred to Immobilon-P (Millipore) membranes, and membranes were immunoblotted with anti-CD19 antibodies using reported procedures (Feilotter, et al. 1994, Nucleic Acids Res. 22:1502-1503; Mahajan, et al. 2001, J. Biol Chem. 276:31216-31228; Uckun, et al. 2010, Proc. Natl. Acad. Sci. USA. 107:2902-2907; Vassilev, et al. 1999, J. Biol Chem. 274:1646-1656). Controls also included whole cell lysates of BCL-1 cells, as indicated.

### Pull-Down Experiments Using MBP-CD19ECD Fusion Protein

Pull-down Experiments Using MBP-CD19ECD Fusion Protein: The cDNA encoding the extracellular domain of human CD19 (CD19ECD) was cloned into the E. coli expression vector pMAL-C2 with the isopropyl-1-thio-β-galactopyranoside-inducible Ptac promoter to create an in frame fusion between the CD19ECD coding sequence and the 3′ end of the E-coli malE gene, which codes for maltose-binding protein (MBP). E. coli strain DH5α was transformed with the generated recombinant plasmid and single transformants were expanded in 5 ml of LB medium (1% tryptone, 1% NaCl, 0.5% yeast extract) containing ampicillin (1000 μg/ml) by overnight culture at 37° C. Expression of the MBP-CD19ECD fusion protein was induced with 10 mM isopropyl-1-thio-β-galactopyranoside.

The cells were harvested by centrifugation at 4500×g in a Sorvall RC5B centrifuge for 10 minutes at 4° C., lysed in sucrose-lysozyme buffer (20 mM Tris, pH 8.0, 150 mM NaCl, 10% sucrose, 1 mM EDTA, 20 mM lysozyme), and further disrupted by sonication. After removal of the cell pellets by centrifugation at 35,000×g for 1 hour at 4° C., MBP-CD19ECD fusion protein was purified from the culture supernatant by amylose affinity chromatography, as previously described (Vassilev, et al. 1999, J. Biol Chem. 274:1646-1656). MBP-CD19ECD was noncovalently bound to amylose beads under conditions of saturating protein, as reported (Vassilev, et al. 1999, J. Biol Chem. 274:1646-1656).

Nonidet P-40 lysates of human T-lineage ALL cell lines MOLT-3 and JURKAT, pre-B ALL cell line NALM-6, and EBV-transformed lymphoblastoid cell line BCL-1 were prepared and 500 μg of each lysate was incubated with 50 μl of MBP-CD19ECD fusion protein coupled to amylose beads for 2 hours on ice. The fusion protein adsorbates were washed with ice-cold 1% Nonidet P-40 buffer and resuspended in reducing SDS sample buffer. Samples were boiled for 5 minutes and then fractionated on SDS-PAGE. Proteins were transferred to Immobilon-P (Millipore Corp) membranes, and membranes were immunoblotted with anti-CD19-L monoclonal antibody.

### Fusion Gene, Protein, Vector

The pFUSE-derived mammalian cell expression vector expressing a rhCD19L:sTRAIL fusion protein (pFUSE-rhCD19L-mCH1-sTRAIL) was constructed for expression of rhCD19L:sTRAIL in CHO cells. The construct contains a full-length cDNA for recombinant (r) human (h) CD19-L, a modified (m) CH1 linker segment encoding a 19-amino acid portion of the human IgG1-CH1 domain, a 0.5-kb sTRAIL cDNA fragment from TRAIL cDNA (pCMV6AC-TRAIL vector SC#321920, Origene, CA) encoding amino acids 114 to 281 of TRAIL protein, and a sequence encoding a 20-amino acids of human IL2 signal peptide to facilitate secretion of the fusion protein. The pFUSE-based backbone vector allows expression of recombinant proteins in CHO cells at μg/mL to mg/mL concentrations. The CHO cell system is one of the most widely used mammalian expression systems for plasma-free manufacturing of FDA-approved recombinant protein therapeutics.

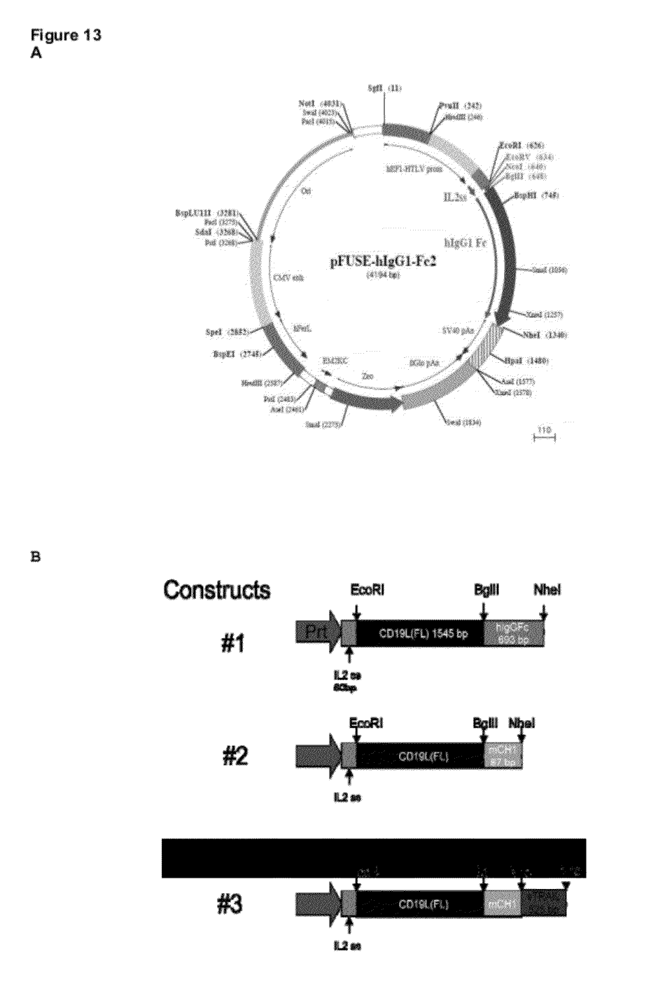

As shown in FIG. 13, the CD19L fusions constructed using the backbone vector pFUSE-hIgGFc (Invitrogene, CA) (FIG. 13, A). A first CD19L fusion construct, Construct#1, CD19L:Fc fusion, was assembled with the coding sequence for the full length CD19-L polypeptide ligated in-frame to the N-terminus of the human IgG Fc fragment. In Construct#2, CD19L:mCH1, the hIgGFc fragment of Construct 1 was replaced with an mCH1 linker. Finally, in Construct#3, the sTRAIL fragment was inserted in-frame to the C-terminus of the mCH1 linker to form the CD19-L:sTRAIL fusion construct (FIG. 13 B).

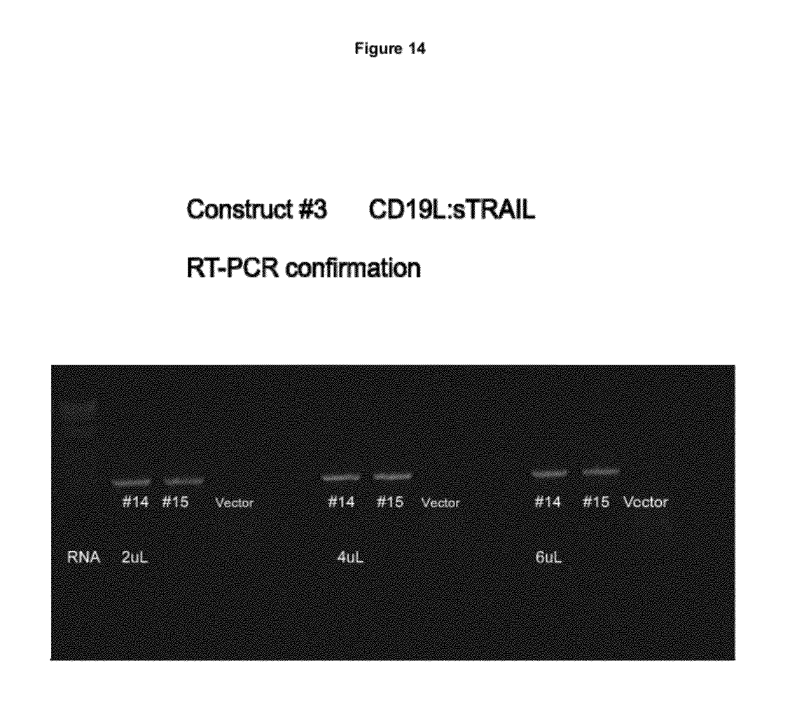

Clones of the pFUSE-CD19L:sTRAIL expression construct were transfected into 293T cells. An empty vector was used as a control (VC). Total RNA was extracted from the transfected cells after a culture period of 24 hours. One-step RT-PCR was performed using the sTRAIL cloning primer set: F: 5′ AGAGAAAGAGGTCCTCAG (SEQ ID NO:8), R: 5′ TTGGGGCCTTTTTAGTTGGCTAA (SEQ ID NO: 9). The expected 0.5 kb PCR product was shown only in the pFUSE-CD19L:sTRAIL transfectants, and not in the VC transfectant, showing successful production of the fusion protein (FIG. 14).

An automated hollow-fiber bioreactor (AcuSyst-Maximizer 1000, Biovest International) can be used in optimization studies under conditions of serum-free, protein-free culture medium formulations, varying concentrations of nutrients for pilot-scale production of rhCD19L:sTRAIL, and using varying numbers of CHO cells in the inoculum (10⁶-10⁹ cells/bioreactor), different control set points for pH, temperature, media circulation and pump speed, cycling volumes, and times. See, for example: Leukemia and Lymphoma 27:275-302, 1997.

Supernatant containing the secreted fusion protein is collected from the extracapillary (EC) circuit of the bioreactor into a harvest bottle using a harvest pump. Production levels are monitored by analysis of collected samples using anti-CD19L and anti-TRAIL ELISA. Supernatants is kept frozen at −80° C. until purification of the CD19-L:sTRAIL fusion protein. The fusion protein is purified by column chromatography and monomers and dimers are separated from trimers by size exclusion. Pro-apoptotic activity of these fractions is analyzed individually, with an expectation that trimers are more potent than monomers and dimmers based on published experience with scFvCD19:sTRAIL fusion protein.

### Generation of Chicken DT40 Lymphoma Cells Expressing Human CD19 Receptor:

Human CD19 cDNA was cloned into an EcoR1 site of the chicken actin promoter-based pApuro expression vector, using published procedures (Takata, et al. 1994, EMBO J. 13:1341-1349). 30 μg of Not1-linearized CD19 cDNA was added to 10×106 DT40 cells in 0.5 ml PBS, pH 7.0, in a 0.4 cm gap-cuvette. Electroporation was done using Bio-Rad gene pulser at 550V, 25 μF. Cells were allowed to recover for 12 hours before selecting in the presence of 0.5 μg/ml puromycin. Selection was done in a 96-well plate to minimize the number of clones generated in each well to one. Expression of the CD19 gene in DT40 clones was confirmed by anti-CD19 immunoblotting.

### Primary Cells

Highly enriched populations of Ficoll-Hypaque-separated leukemia cells isolated from surplus bone marrow specimen remnants of patients with newly diagnosed or relapsed ALL as well as cryopreserved normal thymocytes from surgical thymus specimens removed from children undergoing thoracic surgery for a cardiac defect were used in the described experiments with approval of the PHI Institutional Review Board (IRB) under the exemption category (45 CFR Part 46.101; Category #4: Existing Data, Records Review, and Secondary Use of Pathologic Specimens) in accordance with DHHS guidelines.

### Immunofluorescence Microscopy, Immunoprecipitation, and Western Blot Analyses

Confocal imaging, immunoprecipitation, and immunoblotting using the ECL detection system (Amersham Pharmacia Biotech) were performed, as described in detail in previous publications (Mahajan, et al. 2001, J. Biol Chem. 276:31216-31228; Uckun, et al. 2010, Proc. Natl. Acad. Sci. USA 107:2902-2907, 2010; Vassilev, et al. 1999, J. Biol Chem. 274:1646-1656).

Deconvolution microscopy was performed using the Deltavision deconvolution system from Applied Precision which consists of a Nikon TE200 inverted microscope outfitted for epifluorescence and fitted with an Applied Precision nanomover stage for precise control of the stage. Image collection was through a Photometrics CH350/L liquid cooled CCD camera. The system was controlled by SoftWoRx imaging software run on a Silicon Graphics O2 workstation.

### Selective Binding of CD19L to CD19ECD Present in B-Lineage Lymphoid Cells

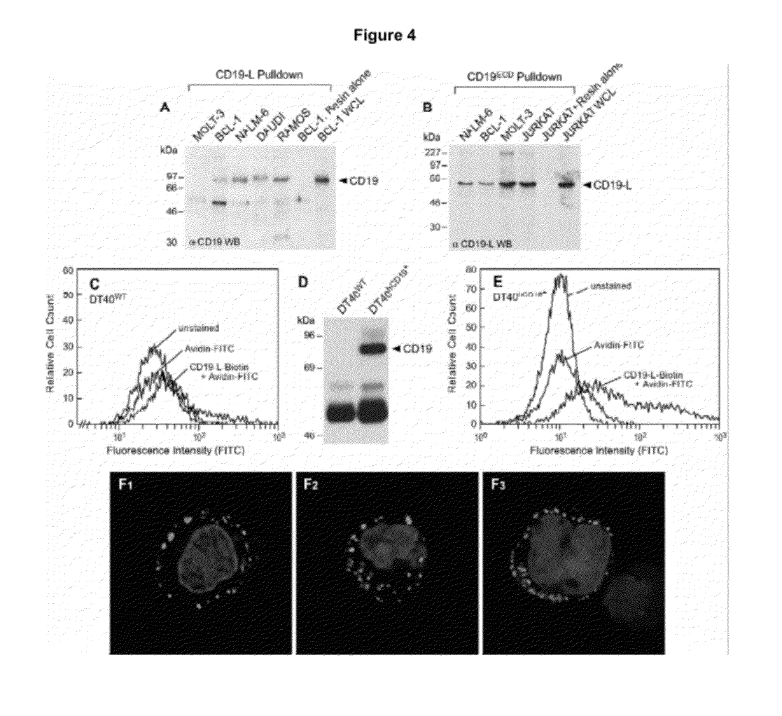

FIG. 4 demonstrates selective binding of CD19-Ligand (CD19-L) to the extracellular domain of CD19 co-receptor (CD19ECD) in human lymphoid cell lines. Binding of recombinant CD19-L coupled with nickel beads to native CD19 co-receptor protein present in lysates of CD19+ human B-lineage lymphoid cell lines was examined. CD19-L associated proteins pulled down from CD19+ B-lineage lymphoid cell lines BCL-1, NALM-6, DAUDI, and RAMOS were immunoblotted with anti-CD19 antibody and demonstrated the presence of the 95-kDa native CD19 co-receptor protein. In contrast, nickel beads without coupled CD19-L did not pull down CD19 co-receptor from BCL-1 lysates. No CD19 was detected in CD19-L complexes pulled-down from CD19-T-lineage ALL cell line MOLT-3 that was included as a negative control (see FIG. 4(A)).

Parallel pull-down experiments used a maltose-binding protein (MBP)-CD19ECD fusion protein. As shown in FIG. 4(B), native CD19-L from human lymphoid cell lines bound to the CD19 extracellular domain (CD19ECD).

Binding of biotin-labeled soluble CD19-L protein to the surface of CD19 co-receptor positive cells was examined using immunofluorescence staining combined with flow cytometry and deconvolution microscopy. Recombinant human CD19-L protein showed specific binding to human CD19 expressed on transfected DT40 chicken B-lymphoma cells, but not to wild-type DT40 cells as shown in FIGS. 4C-E. Likewise, recombinant CD19-L protein exhibited strong surface binding to CD19+ primary human B-lineage ALL cells, as shown in FIGS. 4(F1-3).

### Example 3

### CD19-L Expression Analysis

### Northern Blot Analysis of CD19-L Expression

A 1.5-kb fragment from the 5′-end of CD19-L cDNA was labeled with α-32P-dCTP using DECAprime II DNA labeling kit (Ambion Inc. TX). Total RNA was isolated from various cell lines (5×107 cells/sample) using the Ultraspec™ RNA isolation system from Biotecx Laboratories Inc. (Houston, Tex.) according to the manufacturer's instructions. Messenger RNA was obtained from the total RNA samples using the magnetic-based mRNA isolation kit from Boehringer Mannheim. Three microgram samples of mRNA were loaded on a 1.1% formamide denaturing agarose gel. After electrophoresis, the mRNA samples were transferred to a Magna nylon membrane (MAGNA, MA) and crosslinked with UV light. Hybridization-ready Multiple-Tissue-Northern (MTN) blot membranes were obtained from Clontech Laboratories, Inc. (www.clontech.com).

Membranes were first prehybridized in Rapid-hyb buffer (Amersham, Ill.) at 65° C. for 1 hour and then hybridized with 5 ng (2×10⁷ cpm) α-32P-dCTP-labeled CD19-L probe in Rapid-hyb buffer at 65° C. for 1.5 hour using previously published procedures (Niehoff, et al. 2000, Radiat Res. 154:145-150). For quality control of the loaded mRNA samples, blots were hybridized with a β-actin probe.

### Immunophenotyping, Immunofluorescence Microscopy, Immunoprecipitation, and Western Blot Analyses

Immunophenotyping, confocal imaging, immunoprecipitation, and immunoblotting using the ECL detection system (Amersham Pharmacia Biotech) were performed, as described in detail in previous publications (Mahajan, et al. 2001, J. Biol Chem. 276:31216-31228; Uckun, et al. 2010, Proc. Natl. Acad. Sci. USA 107:2902-2907; Vassilev, et al. 1999, J. Biol Chem. 274:1646-1656; Uckun, et al. 1988, Blood 71:13-29). Cell fractionation into nuclear and cytoplasmic fractions was achieved using published procedures (Hasegawa, et al., 2009, British Journal of Cancer 100:1943-1948) and the Nuclear Extract Kit from Invitrogen. Deconvolution microscopy was performed as described in Example 2.

### CD19-L is Expressed in T-Lineage Cells

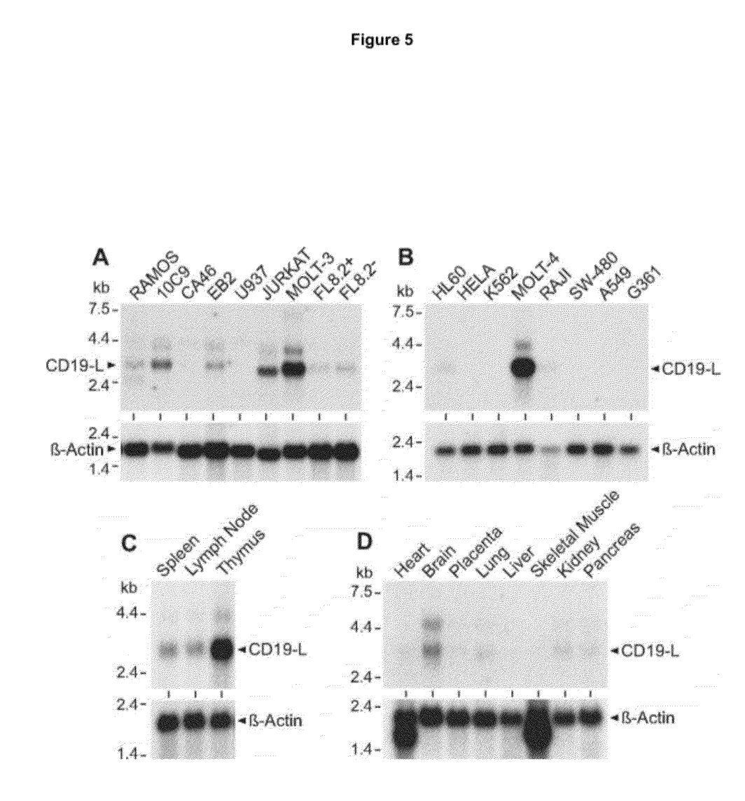

As shown in FIG. 5, a 3-kb transcript of CD19-L was detected in the T-lineage ALL cell lines MOLT-3, MOLT-4, and JURKAT, in the B-lineage leukemia/Burkitt's lymphoma cell lines RAMOS and EB2, in the AIDS-associated EBV lymphoma cell line 10C9, and in the fetal liver derived normal lymphocyte precursor cell lines FL8.2+ (Pro-B/T) and FL8.2-(Pro-B). A minor hybridizing band of 4.4-kb was also detected in some of the cell lines that expressed the 3-kb major CD19-L mRNA species.

Some cell lines did not express CD19-L mRNA. The Burkitt's lymphoma cell lines (RAJI and CA46), U937 histiocytic lymphoma cell line, K562 CML/erythroleukemia cell line, and HL60 AML cell line did not express CD19-L mRNA. These results, shown in FIG. 5, provide evidence that CD19-L gene expression is limited to the lymphocyte compartment of the human lymphohematopoietic system and is particularly abundant in T-lineage cells.

CD19-L transcript was detected in mRNA samples from spleen, lymph node, and thymus. However, CD19-L transcript was not detected in any of the non-lymphohematopoietic solid tumor cell lines tested, including HELA (cervical cancer), SW-480 (colorectal cancer), A549 (lung cancer), and G361 (malignant melanoma). Outside the lymphohematopoietic system, the only organ tissue that showed labeling by the CD19-L cDNA probe was the brain. CD19-L transcript was not detected in heart, placenta, lung, liver, skeletal muscle, kidney, pancreas (FIG. 5).

### CD19L Detected in Both T and B Lineage Lymphoid Cells

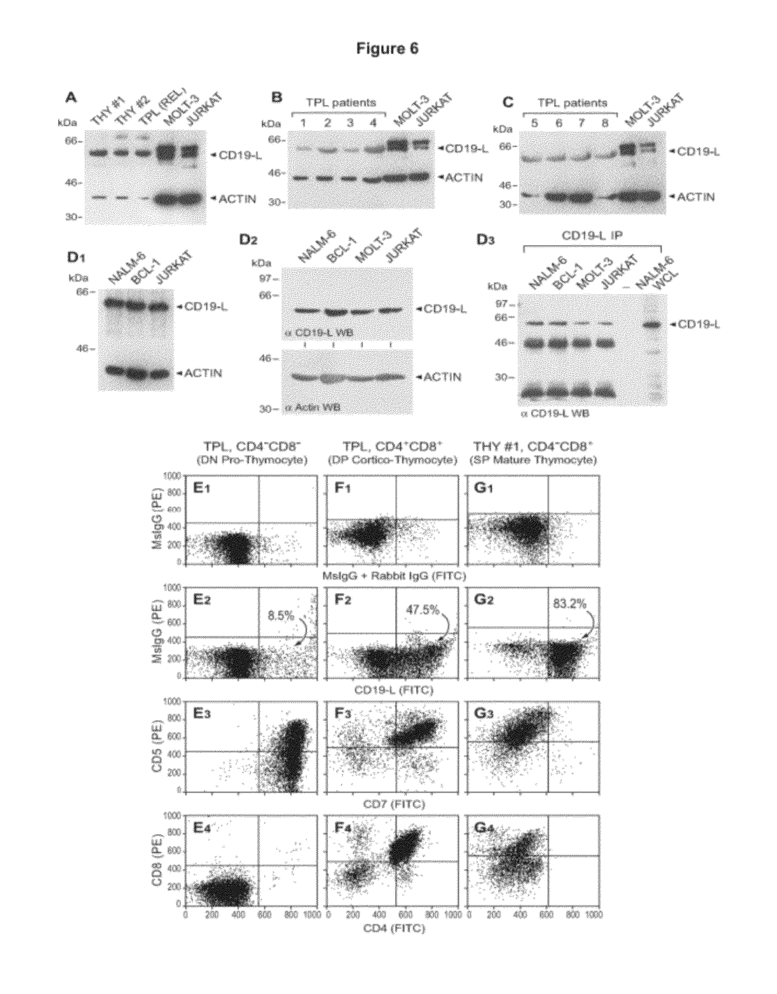

As shown in FIG. 6, CD19-L was detected in a variety of whole cell lysates obtained from both T lineage and B lineage lymphoid cells. CD19-L protein was detected by Western blot analysis in whole cell lysates from T-lineage lymphoid cells, including thymocytes, T-lineage ALL cell lines and primary leukemic T-cell precursors from T-lineage ALL patients, as well as in B-lineage lymphoid cells, including NALM-6 pre-B ALL cell line, and BCL-1 mature B-cell line. CD19-L protein was also detected by Western blot analysis in whole cell lysates from NALM-6 pre-B ALL, BCL-1 mature B-cell, MOLT-3 and JURKAT T-lineage ALL cell lines. CD19-L protein was detected as a 54-kDa protein by Western blot analysis in whole cell lysates of thymocytes as well as in T-lineage ALL cell lines and primary leukemic T-cell precursors from newly diagnosed pediatric T-lineage ALL/T-precursor leukemia (TPL) patients. (FIG. 6 A-D3)

Native CD19-L protein was immunoprecipitated using a anti-CD19-L mouse monoclonal antibody from whole cell lysates of NALM-6 pre-B ALL, BCL-1 mature B-cell, MOLT-3 and JURKAT cell lines. CD19-L immune complexes were subjected to Western blot analysis with anti-CD19-L monoclonal antibody. A distinct CD19-L protein band was detected in the anti-CD19-L antibody immunoprecipitates from each cell line. (FIG. 6)

CD19-L Gene Expression Differs from TOX1 Expression

Expression of the CD19-L gene is limited to the lymphocyte compartment within the human lymphohematopoietic system and is particularly abundant in T-lineage cells. The expression profile of CD19-L differs from the TOX1 expression profile at least in its lack of expression in liver and abundant expression in spleen and B-lineage lymphoid cells, as shown in FIG. 5. While TOX1 is abundantly expressed in mature T-cells but shows low-level expression in immature T-cell precursors, CD19-L displays abundant expression in thymocytes as well as leukemic T-cell precursors from T-lineage ALL patients corresponding to immature DN pro-thymocyte and DP cortico-thymocyte stages of human T-cell ontogeny (FIG. 6) and T-lineage ALL cell lines MOLT-3 and JURKAT (see FIGS. 5, 6, and 7).

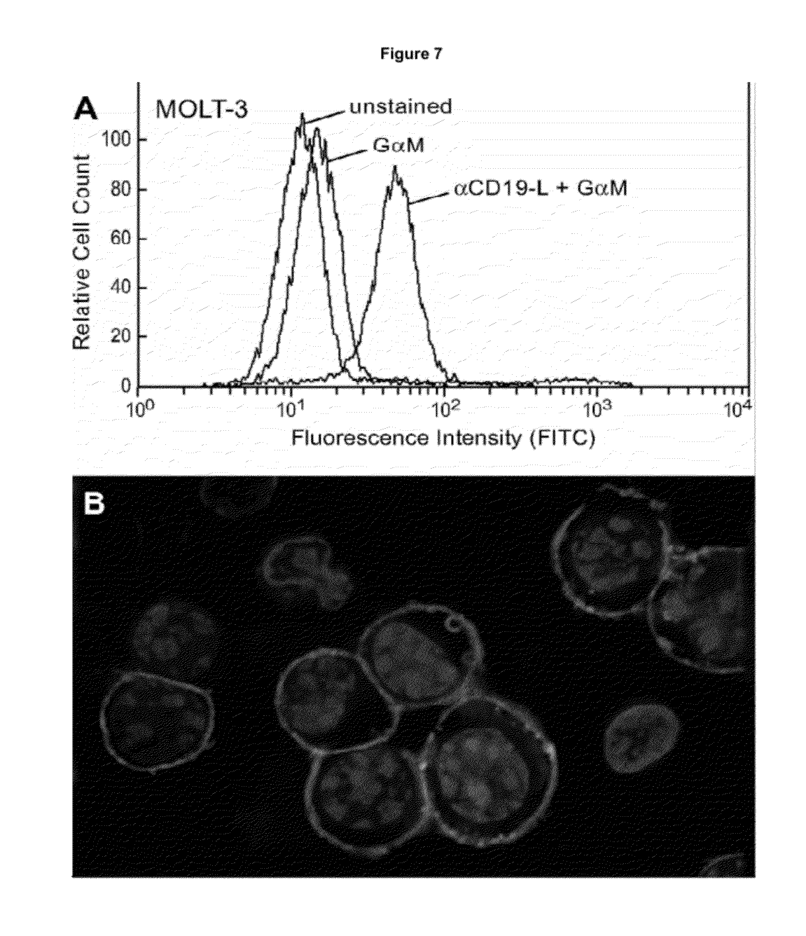

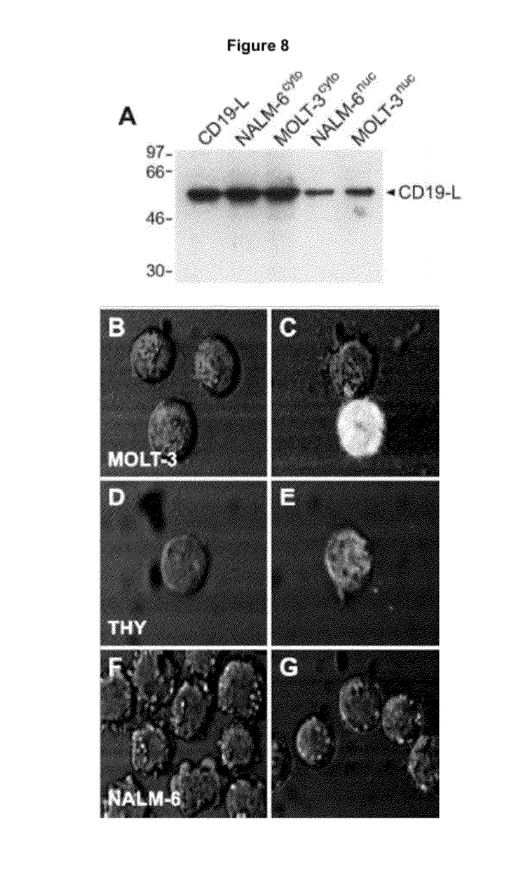

B-cells and B-cell precursors lack TOX1 that exhibits specificity for T-lineage lymphoid cells within the lymphoid compartment (see Rhodes D R, et al. 2007, Neoplasia. 9:166-180). In contrast to TOX1, CD19-L is expressed in the B-lineage lymphoid cells at all stages of human B-cell ontogeny, including FL8.2+ fetal liver derived biphenotypic CD2+CD19+ pro-B/T cells, FL8.2-fetal liver-derived pro-B cells, NALM-6 pre-B cells and BCL-1 EBV-transformed mature B-cells (FIGS. 2, (A), and 6(D.1)). TOX1 expression is limited to the nucleus (Uckun, et al., 1988, Blood. 71:13-29; Uckun, et al. 1996, Science. 273:1096-1100; Rhodes, et al. 2007, Neoplasia. 9:166-180), while CD19-L is expressed in the nucleus, cytoplasm, and on the surface membrane (FIG. 7, FIG. 8). CD19-L nuclear staining was observed in MOLT-3 cells (FIG. 8 (Panels B, C)) and thymocytes (THY) (FIG. 8 (Panels D, E)), whereas NALM-6 cells exhibited a perinuclear cytoplasmic staining pattern (FIG. 8 (Panels F, G)).

### Example 4

### CD19-L Functional Analysis

### Gene Expression Profiling

RNA was isolated from NALM-6 cells after 24 hours treatment with CD19-L (100 ng/mL) using the ArrayGrade Total RNA isolation kit (SuperArray, SA Biosciences, Frederick, Md.). Control treatment was with PBS. cDNA was synthesized from the isolated total RNA by an in vitro linear polymerase reaction using the TrueLabeling Linear RNA Amplification kit (SuperArray). cDNA was used to prepare amplified biotin-labeled cRNA, which was purified using cRNA Cleanup kit from SuperArray.

The cRNA was hybridized to 4 panels of oligo microarrays (SuperArray Bioscience Corporation) (www. SABiosciences.com), (1) GEArray S Series Human Immunology Signaling Pathways Gene Array (HS-605; 416 genes); (2) Oligo GEArray Human Hematopoietic Stem Cells and Hematopoiesis Microarray (OHS-054; 128 genes); (3) GEArray® Express Human Apoptosis Microarray (EHS-012; 128 genes); and (4) GEArray Express Human Autoimmune and Inflammatory Response Microarray (EHS-803; 480 genes). Normalization of expression values for each panel of genes was achieved by dividing the expression value of each gene by the mean of genes between 25th and 75th percentiles.

A bivariate scatter plot was performed for each control sample against the CD19-L treated NALM-6 sample stratified by panel, cell line and time point to assess dispersion of normalized values around the line of unity. T-tests with degrees of freedom correction for unequal variances (Excel formula) were performed on normalized values to identify discriminating genes between comparison subsets. True Discovery Rates (TDR) were calculated from assessing the number of genes that were expected to change at 3 P-value thresholds (E) and the number of changes that were observed (O) to be at or greater than the p-value threshold (TDR=100*((1−E)/O)).

One hundred and fifty genes were represented on two or more panels out of the four panels screened (EHS803; HS605; OHS054; EHS012). Since the expression value for each gene in each panel was derived from normalization relative to the mean expression value of the genes between 25 and 75 percentiles on each panel, the normalized expression values for any one gene across the 4 panels had different expression values depending on the panel the gene was interrogated.

A T-test (two-sample, unequal variance) compared the effect of CD19-L treatment using the standardized values across all panels and ranked according to P-value for NALM-6 cells at 24 hours. Most discriminating genes for CD19-L treatment of NALM-6 cells were cross-referenced to the Oncomine™ Research Data Base (http://www.oncomine.org/) for leukemia and lymphoma studies.

Meta-analysis was used to interrogate each of the identified signature genes for its previously reported expression values and associations in other leukemia or lymphoma studies. For each gene the fold difference and T-test p-value are reported for log-transformed, normalized expression levels.

### CD19-L Treatment Alters Expression of Apoptosis-Regulating Genes

Treatment of the B-lineage ALL cell line NALM-6 with 100 ng/mL CD19-L for 24 hours altered the regulation of gene expression and altered the expression levels of 13 genes directly involved in regulation of apoptosis. Six genes were up-regulated and seven genes were down-regulated in response to treatment with CD19-L treatment.

| Up-regulated Genes | Down-regulated Genes |
|---|---|
| TNFSF7 | IRF4 |
| SPP1 | MAPK11 |
| TNFSF18 | TNFRSF7/CD27 |
| FLT3LG | ETS1 |
| HDAC5 | YY1 |
| NFKB1 | IRAK2 |
| | PAX5 |

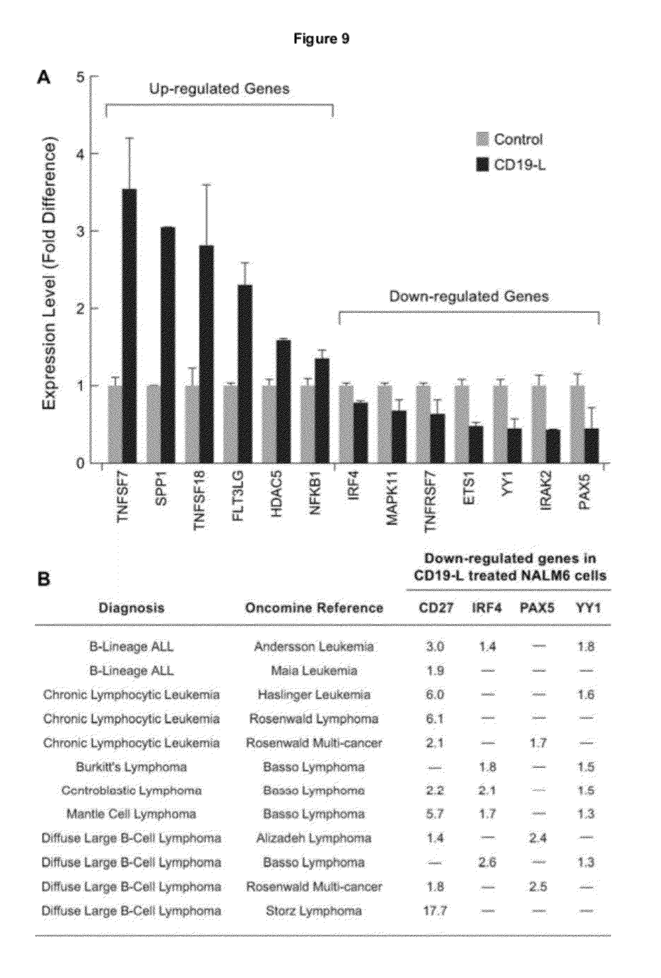

Meta-analysis of four genes significantly down-regulated by CD19-L, TNFSF7/CD27, IRF4, PAX5, and YY1, was conducted using the Oncomine™ Database and were interrogated by using the database for reported expression in other leukemia/lymphoma studies. Results are shown in FIG. 9, and are expressed as “fold difference” relative to normal expression (T-test p-values <0.05). Each of the four genes was expressed in malignant cells from patients with lymphoid malignancies at significantly higher levels than in normal lymphocyte controls, suggesting that these genes promote a very aggressive in vivo growth of patients' primary neoplastic cells causing disseminated overt leukemia/lymphoma. The most enriched gene that showed reduced expression levels after CD19-L treatment was the anti-apoptotic TNFRSF7/CD27 gene. This gene was reported to be up-regulated in B-lineage lymphoid neoplasms in 10 of 12 studies (FIG. 9).

These data provide putative biomarkers for lymphoid disease likely to be susceptible to therapeutic treatment by CD19-L, for example by analyzing a subject's sample for a genomic profile that indicates the presence or increase in the anti-apoptosis related genes IFR4, MAPK11, TNFRSF7/CD27, ETS1, YY1, IRAK2, PAK5, and/or for the absence or decrease of one or a plurality of the apoptosis related genes TNFSF7, SPP1, TNFSF18, FLT3LG, HDAC5, NFKB1. In an embodiment, a biomarker panel for selecting a subject suitable for treatment of leukemia and/or lymphoma with CD19-L comprises one or a plurality of the genes, TNFSF7/CD27, IRF4, PAX5, and YY1.

The data also provides a basis for identifying aggressive leukemia and/or lymphoma by analyzing a subject's sample for increased expression versus a normal lymphocyte control of one or a plurality of the following four genes: CD19-L, TNFSF7/CD27, IRF4, PAX5, and YY1.

Methods and test kits providing amplification and/or hybridization probes for the analysis of the gene profiles discussed above are provided herein.

### Example 5

### Treatment with CD19-L Induces Apoptosis

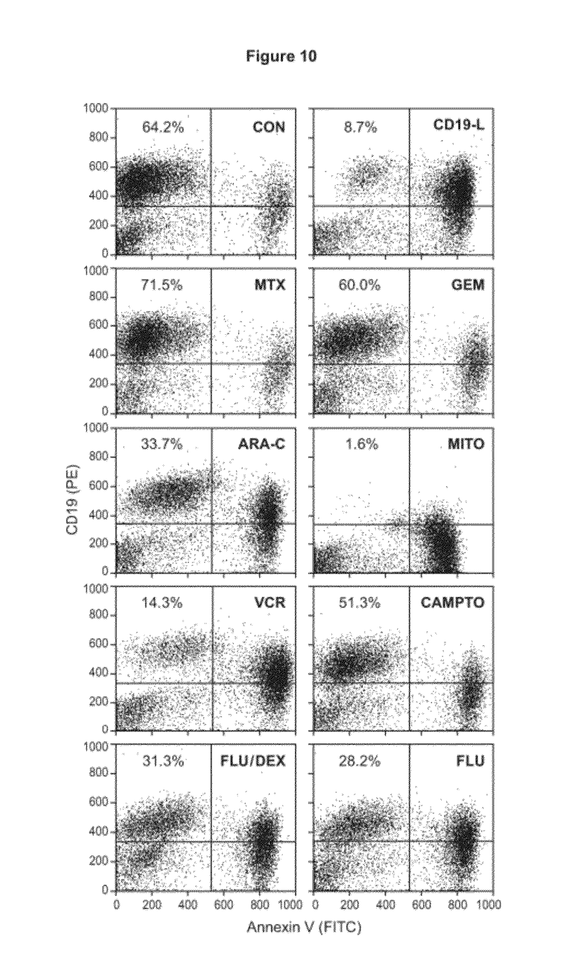

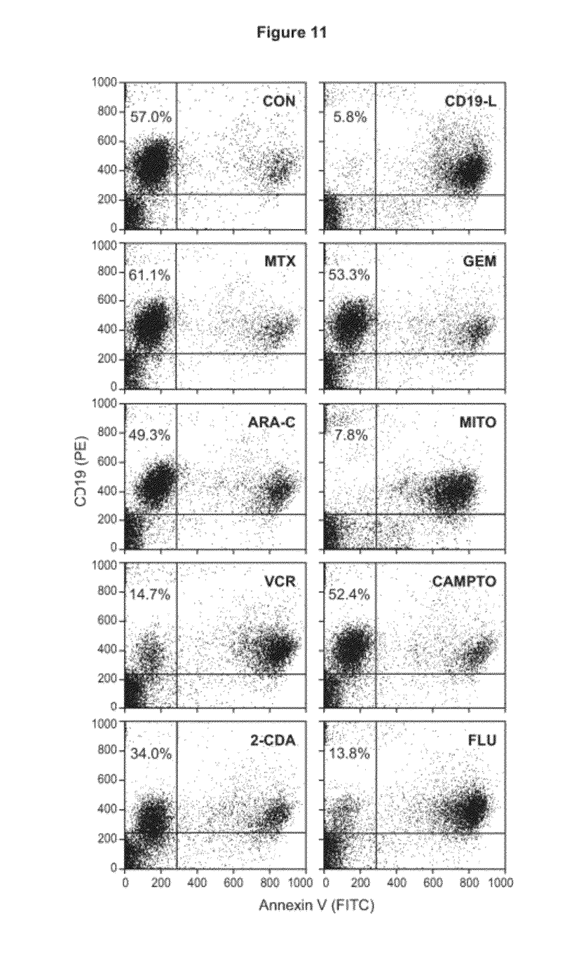

CD19-L is was analyzed to for potential therapeutic effect on leukemic cells and was found to induce apoptosis in primary leukemic cells obtained from relapsed B-Lineage ALL patients, independent of their resistance to other drugs, as show in FIG. 10. Each drug was used at 25 μg/mL except DEX, used at 10 μg/mL. The substantial decrease in the number of CD19+Annexin-V− viable leukemia cells with the concomitant increase of the percentage of CD19+Annexin-V+ apoptotic leukemia cells after 24 hours of exposure to CD19-L shown in FIG. 10 confirms the induction of apoptosis by CD19-L. Quantitative flow cytometric apoptosis assays from a second representative sample show that CD19-L (5 μg/mL) further confirms CD19-L induction of apoptosis in primary leukemic cells independent of their resistance to other drugs (each used at 25 μg/mL), as documented by the substantial decrease in the numbers of CD19+Annexin-V− viable leukemia cells with concomitant increase of the percentage of CD19+Annexin-V+ apoptotic leukemia cells after 24 hours of exposure (FIG. 11). For apoptosis assays, a standard flow cytometric method as described in Uckun et al., 2006 Brit J Haematol. 135: 500-508) was used.

These results demonstrate that CD19-L promotes apoptosis-related signaling and alters gene expression in CD19+ human leukemia cells.

It will be apparent to one of ordinary skill in the art that various modifications of the materials and methods for practicing the invention can be made. Such modifications are to be considered within the scope of the invention as defined by the following claims.

Each of the references from the patent and public literature cited herein is hereby expressly incorporated in its entirety by such citation.

1. An isolated polypeptide consisting of a polypeptide having the amino acid sequence of SEQ ID NO:2 or an active fragment thereof capable of binding the CD19 extracellular domain.

2. A polynucleotide encoding the polypeptide of claim 1.

3. The polynucleotide of claim 2, having nucleic acid sequence of SEQ ID NO:1.

4. A composition comprising a polypeptide of claim 1.

5. A fusion molecule comprising the polypeptide of claim 1.

6. The fusion molecule of claim 5, further comprising an chemotoxic or chemostatic molecule, a radionucleotide, or a therapeutic polypeptide.

7. The fusion molecule of claim 5, further comprising a molecule targeted for delivery to a T-cell.

8. The fusion molecule of claim 5, further comprising a cytokine.

9. The fusion molecule of claim 8, wherein the cytokine is IL2, IL7, or TNF.

10. The fusion molecule of claim 5, further comprising a detection marker.

11. The fusion molecule of claim 10, wherein said detection marker is a FLAGG, poly-HIS, GST, or MBP tag.

12. The fusion molecule of claim 10, wherein said detection marker is an immuno-fluorescent, chemi-luminescent or colorimetric marker.

13. The fusion molecule of claim 5, further comprising an antibody or ligand capable of binding a T-cell antigen.

14. A fusion molecule comprising the polynucleotide of claim 2.

15. The fusion molecule of claim 14, further comprising a polynucleotide expressing a polypeptide targeted to T-cells.

16. A transgenic cell comprising a polynucleotide of claim 2.

17. The transgenic cell of claim 16, wherein said polynucleotide has the nucleic acid sequence of SEQ ID NO:1.

18. A method for inducing apoptosis in a CD19 expressing cell comprising contacting said cell with a composition comprising the polypeptide of claim 1, a polynucleotide encoding the polypeptide of claim 1, or a fusion molecule comprising said polypeptide or polynucleotide.

19. The method of claim 18, wherein said polypeptide is a recombinant, soluble polypeptide.

20. The method of claim 18, wherein said fusion molecule comprises a molecule targeted for delivery to a T-cell.

21. The method of claim 18, wherein said contacting comprises contacting said cell with composition comprising a polynucleotide encoding a polypeptide having the amino acid sequence of SEQ ID NO:2.

22. A method for treating a subject suffering from a disease characterized by CD19 expressing lymphoid cells comprising administering to the subject a composition comprising the polypeptide of claim 1 or a fusion molecule comprising said polypeptide.

23. The method of claim 22, wherein said administered composition comprises a polynucleotide encoding the polypeptide having the amino acid sequence of SEQ ID NO:2 or a fusion molecule of said polypeptide.

24. The method of claim 18, wherein said CD19 expressing cell is a B-lineage leukemia lymphoma cell.

25. The method of claim 18, wherein said CD19 expressing cell is a B-lineage ALL cell.

26. The method of claim 22, wherein said subject was previously treated for ALL with a chemotherapeutic drug and relapsed.

27. The method of claim 22, wherein said subject demonstrates resistance to one or more therapeutic drug used to treat ALL.

28. The method of claim 22, wherein said subject demonstrates resistance to one or more of methotrexate, gemcitabine, cytarabine, mitoxantrone, vincristine, campothothecin, cladribine, and fludarabine.

29. A method for treating a subject suffering from a disease characterized by CD19-L expressing lymphoid cells comprising administering to the subject a composition comprising a CD19 antigen polypeptide, a fragment thereof that retains the ability to bind CD19-L, or a fusion molecule that comprises said polypeptide or fragment.

30. The method of claim 29, wherein said administering comprises administering a fusion molecule comprising the CD19 antigen polypeptide or a fragment thereof.

31. The method of claim 30, wherein said administering comprises administering a polynucleotide encoding the CD19 antigen polypeptide, fragment, or fusion.

32. The method of claim 29, wherein said CD19-L expressing cell is a T-lineage leukemia or lymphoma cell.

33. The method of claim 29, wherein said CD19-L expressing cell is a T-lineage ALL cell.

34. The method of claim 29, wherein said subject was previously treated for ALL with a chemotherapeutic drug and relapsed.

35. The method of claim 29, wherein said subject demonstrates resistance to one or more therapeutic drug used to treat ALL.

36. A method for diagnosing the presence or progression of a disease characterized by CD19 expressing lymphoid cells in a subject, the method comprising:
a) analyzing a sample obtained from the subject to determine the presence or amount of CD19 or CD19 expressing lymphoid cells; and
b) correlating the presence or increased amount of CD19 or CD19 expressing lymphoid cells with the presence or progression of B-cell lymphoid disease; or
c) correlating the absence or reduced amount of CD19 or CD19 expressing lymphoid cells with the absence or regression of B-cell lymphoid disease.

37. The method of claim 36, wherein said analyzing comprises contacting said sample with a composition comprising CD19-L to detect said CD19.

38. The method of claim 37, wherein said CD19-L is linked to a detection tag.

39. An antibody that specifically binds a CD19-L polypeptide of claim 1.

40. An antibody that specifically binds a CD19-L polypeptide encoded by the polynucleotide of claim 2.

41. The method of claim 37, wherein said analyzing comprises further contacting said sample with an anti-CD19-L antibody that specifically binds a CD19-L polypeptide having the amino acid sequence of SEQ ID NO:2.

42. The method of claim 36, wherein said CD19 is soluble or is expressed on the surface of a cell.

43. A method for diagnosing the presence or progression of a T-cell lymphoid disease in a subject, the method comprising:
a) analyzing a sample obtained from the subject to determine the presence or amount of the CD19-L polypeptide or CD19-L expressing lymphoid cells of claim 1; and
b) correlating the presence or increased amount of the CD19-L or CD19-L expressing lymphoid cells with the presence or progression of T-cell lymphoid disease; or
c) correlating the absence or reduced amount of the CD19-L or CD19-L expressing lymphoid cells with the absence or regression of T-cell lymphoid disease.

44. The method of claim 43, wherein said analyzing comprises contacting said sample with a composition comprising CD19 antigen to detect said CD19-L.

45. The method of claim 44, wherein said CD19 is linked to a detection tag.

46. The method of claim 43, wherein said analyzing comprises further contacting said sample with an anti-CD19 antibody.

47. The method of claim 43, wherein said CD19-L is soluble or is expressed on the surface of a cell.

48. A method for treating autoimmune disease in a subject, comprising:
administering to the subject one or a combination of:
a) the CD19-L polypeptide of claim 1;
b) a CD19 polypeptide;
c) an anti-CD19-L antibody; and
d) an anti-CD19 antibody,
or a combination thereof;
wherein said administering disrupts interaction of T-cells and B-cells to inhibit immune reaction.

49. The method of claim 48, wherein said autoimmune disease is graft versus host disease (GVHD), rheumatoid arthritis, inflammatory bowel disease, or organ transplant rejection.

50. A method for predicting a likelihood of a subject's response to CD19-L therapy comprising:
a) analyzing a sample obtained from a leukemia/lymphoma patient for the expression of one or a plurality of the following genes: IFR4, MAPK11, TNFRSF7/CD27, ETS1, YY1, IRAK2, PAK5, TNFSF7, SPP1, TNFSF18, FLT3LG, HDAC5, NFKB1; and
b) correlating the presence or increase in one or more of IFR4, MAPK11, TNFRSF7/CD27, ETS1, YY1, IRAK2, PAK5, or the absence or decrease of one or a plurality of TNFSF7, SPP1, TNFSF18, FLT3LG, HDAC5, NFKB1, or a combination thereof with probable response to CD19-L therapy.

51. The method of claim 36, where the following genes are analyzed: TNFSF7/CD27, IRF4, PAX5, and YY1.

52. A method for predicting aggressive leukemia or lymphoma disease in a subject, comprising:
a) analyzing a sample obtained from the subject for the expression of one or a plurality of the following genes: TNFSF7/CD27, IRF4, PAX5, and YY1; and
b) correlating the presence or increase in expression one or more of TNFSF7/CD27, IRF4, PAX5, and YY1 relative to control with a likelihood of aggressive leukemia/lymphoma disease in the subject.

53. A method for treating a leukemia/lymphoma in a subject comprising:
administering the CD19-L polypeptide of claim 1 to a subject having lymphoid cells demonstrating increased expression of one or more of IFR4, MAPK11, TNFRSF7/CD27, ETS1, YY1, IRAK2, PAK5, or decreased expression of one or a plurality of TNFSF7, SPP1, TNFSF18, FLT3LG, HDAC5, NFKB1, or a combination thereof.

54. The method of claim 52, wherein the subject demonstrates increased expression of one or more of TNFSF7/CD27, IRF4, PAX5, and YY1 relative to control.

55. The method of claim 52, wherein said disease is leukemia/lymphoma, graft versus host disease (GVHD), autoimmune disorders, rheumatoid arthritis, inflammatory bowel disease, or organ transplant rejection.

56. The method of claim 52, wherein said disease is ALL.

57. A process for producing a CD19-L polypeptide, comprising:
a) inserting an expression vector comprising the polynucleotide of claim 2 into a cell;
b) expressing the polypeptide in said cell; and
c) optionally purifying the polypeptide from the cell.

58. The process of claim 57, wherein the cell is a bacterial, baculovirus, or mammalian cell.

59. The process of claim 57, wherein the cell is an E. coli cell.

60. The process of claim 57, wherein the cell is an immortalized mammalian cell line.

61. (canceled)
